(19) United States
(12) Patent Application Publication
(10) Pub. No.: US 2006/0204961 A1
(43) Pub. Date: Sep. 14, 2006

(54) Reagents and methods for diagnosis of attention deficit hyperactivity disorder

(76) Inventors: James M Swanson, Irvine, CA (US); Robert K Moyzis, Corona Del mar, CA (US)

Correspondence Address: CHRISTIE, PARKER & HALE, LLP, PO BOX 7068, PASADENA, CA, 91109-7068, US

(21) Appl. No.: 10/539,082

(22) PCT Filed: Dec. 29, 2003
(86) PCT No.: PCT/US03/41409

§ 371 Date: Dec. 16, 2005

Related U.S. Application Data
(60) Provisional application No. 60/436,903, filed on Dec. 26, 2002.

(51) Int. Cl. C12Q 1/68; C07H 21/04

(52) U.S. Cl. 435/6; 536/24.3

(57) ABSTRACT

The subject invention provides reagents and methods for diagnosing Attention Deficit Hyperactivity Disorder (ADHD) in an individual based on the presence of specific alleles of the DRD4 gene or other markers found within a region of strong linkage disequilibrium to the DRD4 7R allele in the DNA of the individual.

Attention Deficit Hyperactivity Disorder (ADHD) is a neurobehavioral disorder defined by symptoms of developmentally inappropriate inattention, impulsivity, and hyperactivity with early onset (American Psychiatric Association DSM-IV: Diagnostic and Statistical Manual of Mental Disorders (Am. Psychiatr. Assoc. Washington, D.C.) 4th Ed. 1994.) Current estimates indicate that 3-6% of school age children are diagnosed with ADHD, making it the most prevalent disorder of childhood (Swanson, J M, Flodman, P, Kennedy, J, Spence, M A, Moyzis, R, Schuck, S, et al., Dopamine genes and ADHD. Neuroscience and Behavioral Reviews 2000; 24, 21-25.) While the broad DSM-IV phenotype of ADHD almost certainly has multiple biological etiologies (Swanson, J, Deutsch, C, Cantwell, D, Posner, M, Kennedy, J L, Barr, C L, et al. Genes and Attention-Deficit Hyperactivity Disorder. Clinical Neuroscience Research 2001; 1, 207-216), numerous family, twin and adoption studies have documented a strong genetic basis (Faraone, S V, Biederman, J. Genetics of attention-deficit hyperactivity disorder. Child Adolesc Clin North Am 1994; 3, 285-291; Faraone, S V, Doyle, A E, Mick, E, and Biederman, J. Meta-analysis of the association between the 7-repeat allele of the dopamine D4 receptor gene and attention deficit hyperactivity disorder. Am J Psychiatry 2001; 158, 1052-1057).

Despite the high heritability of ADHD, initial genome scan-studies have failed to identify genes of major effect (Fisher, S E, Franks, C, McCracken, J T, McGough, J J, Marlov, A J, MacPhie, I L, et al. A genomewide scan for loci involved in Attention-Deficit/Hyperactivity Disorder. Am J Hum Genet 2002; 70, 1183-1196), although a region on chromosome 16p13 has been implicated in subsequent studies by the same group (Smalley, S L, Kustanovich, V, Minassian, S L, Stone, J L, Ogdie, M N, McGough, J J, et al. Genetic linkage of attention-deficit/hyperactivity disorder on chromosome 16p13, in a region implicated in autism. Am J Hum Genet 2002; 71, 959-963.) Such negative results are not unexpected for a complex genetic disorder like ADHD, where phenotypic heterogeneity is likely, and the practical but (to date) restricted sample sizes limit statistical power (Risch, N and Merikangas, K. The future of genetic studies of complex human diseases. Science 1996; 273, 1516-1517; Terwilliger, J D and Weiss, K M. Linkage disequilibrium mapping of complex disease: fantasy or reality? Current Opin Biotechnology 1998; 9, 578-594; Weiss, K M and Terwilliger, J D. How many diseases does it take to map a gene with SNPs? Nature Genetics 2000; 26, 151-157; Zwick, M E, Cutler, D J, and Chakravarti, A. Patterns of genetic variation in mendelian and complex traits. Ann Rev Genomics Hum Genet 2000; 1, 387-407; Sklar, P. Linkage analysis in psychiatric disorders: the emerging picture. Ann Rev Genomics Hum Genet 2002; 3, 371-413.) Candidate gene studies, on the other hand, require much smaller sample sizes to achieve the same statistical power. The efficacy of a dopamine agonist drug, methylphenidate, in the treatment of ADHD has suggested that genes in the dopamine pathway may be involved in the disorder's etiology (Volkow, N D, Wang, G J, Fowler, J S, Logan, J, Franceschi, D, Maynard, L, et al. Relationship between blockade of dopamine transporters by oral methylphenidate and the increases in extracellular dopamine: therapeutic implications. Synapse 2002; 43, 181-187). This dopamine hypothesis of ADHD suggests a number of candidate genes that could logically be tested for their association with the disorder. The draft human genome sequence (International Human Genome Sequencing Consortium, Initial sequencing and analysis of the human genome. Nature 2001; 409, 860-921; Riethman, H C, Xiang, Z, Paul, S, Morse, E, Hu, X-L, Flint, J, Chi, H-C, Grady, D L, and Moyzis, R K. Integration of telomeric sequences with the draft human genome sequence. Nature 2001; 409, 948-951; Cowan, W M, Kopnisky, K L, and Hyman, S E. The Human Genome Project and its impact on Psychiatry. Annu Rev Neurosci 2002; 25, 1-50) has provided information sufficient to examine multiple candidate genes in parallel, often representing most of the proteins in a relevant biochemical pathway.

One of these candidate genes, DRD4 (Van Tol, H H M, Bunzow, J R, Guan, H-C, Sunahara, R K, Seeman, P, Niznik, H B and Civelli, O. Cloning of the gene for a human dopamine D4 receptor with high affinity for the antipsychotic clozapine. Nature 1991; 350, 610-614), located near the telomere of chromosome 11p, is one of the most variable human genes known (Lichter, J B, Barr, C L, Kennedy, J L, VanTol, H H M, Kidd, K K, and Livak, K J. A hypervariable segment in the human dopamine receptor D4 (DRD4) gene. Human Molecular Genetics 1993; 2, 767-773; Chang, F-M, Kidd, J R, Livak, K J, Pakstis, A J, and Kidd, K K. The world-wide distribution of allele frequencies at the human dopamine D4 receptor locus. Hum Genetics 1996; 98, 91-101; Ding, Y-C, Wooding, S, Harpending, H C, Chi, H-C, Li, H-P, Fu, Y-X et al. Population structure and history in East Asia. Proc Natl Acad Sci USA 2000; 97, 14003-14006; Ding, Y-C, Chi, H C, Grady, D L, Morishima, A, Kidd, J R, Kidd, K K et al. Evidence of positive selection acting at the human dopamine receptor D4 gene locus. Proc Natl Acad Sci USA 2002; 99, 309-314.)

What is needed are genetic marker(s) useful in the diagnosis of ADHD, and methods for using the same.

### SUMMARY OF THE INVENTION

The present invention provides a reagent useful for diagnosing attention deficit hyperactivity disorder (ADHD), comprising a polynucleotide corresponding to an allele of DRDR associated with individuals exhibiting ADHD.

The present invention further provides a reagent useful for diagnosing ADHD, comprising a polynucleotide corresponding to the DRD4 7R allele.

The present invention further provides a reagent useful for diagnosing ADHD, comprising a polynucleotide corresponding to a marker the locus of which is within a block of linkage disequilibrium surrounding the DRD4 7R allele.

The present invention further provides a reagent useful for diagnosing ADHD, comprising a pair of oligonucleotides corresponding to an allele of DRDR associated with individuals exhibiting ADHD.

The present invention further provides a reagent useful for diagnosing ADHD, comprising a pair of oligonucleotides corresponding to the DRD4 7R allele.

The present invention further provides a reagent useful for diagnosing ADHD, comprising a pair of oligonucleotides corresponding to a marker the locus of which is within a block of linkage disequilibrium surrounding the DRD4 7R allele.

The present invention further provides a method for diagnosing ADHD in an individual, comprising the steps of:

a) obtaining a tissue sample from the individual;

b) treating the sample so as to expose DNA present in the sample;

c) contacting the exposed DNA with a labeled DNA oligomer under conditions permitting hybridization of the DNA oligomer to any DNA complementary to the DNA oligomer present in the sample, the DNA complementary to the DNA oligomer containing the DRD4 7R allele;

d) removing unhybridized, labeled DNA oligomer; and

e) detecting the presence of any hybrid of the labeled DNA oligomer and DNA complementary to the DNA oligomer present in the sample, thereby detecting and diagnosing ADHD.

The present invention further provides a method for diagnosing ADHD in an individual, comprising the steps of:

a) obtaining a tissue sample from the individual;

b) treating the sample so as to expose DNA present in the sample;

c) contacting the exposed DNA with a labeled DNA oligomer under conditions permitting hybridization of the DNA oligomer to any DNA complementary to the DNA oligomer present in the sample, the DNA complementary to the DNA oligomer containing a marker within a region of strong linkage disequilibrium to the DRD4 7R allele;

d) removing unhybridized, labeled DNA oligomer; and

e) detecting the presence of any hybrid of the labeled DNA oligomer and DNA complementary to the DNA oligomer present in the sample, thereby detecting and diagnosing ADHD.

The present invention further provides a method for diagnosing ADHD in an individual, comprising the steps of:

a) obtaining a tissue sample from the individual;

b) providing an oligonucleotide complementary to the sense strand of the DRD4 gene;

c) providing an oligonucleotide complementary to the antisense strand of the DRD4 gene;

d) treating the sample so as to expose DNA present in the sample;

e) contacting the exposed DNA with the oligonucleotides under conditions permitting amplification of the DRD4 gene;

f) sequencing the product of the amplification; and

g) detecting the presence of the DRD4 7R allele in the sample, thereby detecting and diagnosing ADHD.

The present invention further provides a method for diagnosing ADHD in an individual, comprising the steps of:

a) obtaining a tissue sample from the individual;

b) providing an oligonucleotide complementary to the sense strand of a marker sequence found in an area of strong linkage disequilibrium with the DRD4 7R allele;

c) providing an oligonucleotide complementary to the antisense strand of the marker sequence;

d) treating the sample so as to expose DNA present in the sample;

e) contacting the exposed DNA with the oligonucleotides under conditions permitting amplification of the marker sequence;

f) sequencing the product of the amplification; and

g) detecting the presence of the marker sequence in the sample, thereby detecting and diagnosing ADHD.

### BRIEF DESCRIPTION OF THE DRAWINGS

FIG. 1 Diagrammatic representation of the human DRD4 gene region. Exon positions are indicated by blocks (yellow: noncoding, orange: coding). The approximate positions of a 120 bp promoter region duplication (blue triangle), an exon 1 12 bp duplication (blue triangle), an exon 3 48 bp VNTR (blue triangle), and two intron 3 SNPs are indicated. 2R through 11R variants of the 48 bp VNTR are indicated below exon 3 (blue), along with their worldwide population frequencies determined by PCR analysis (Chang, F.-M., Kidd, J. R., Livak, K. J., Pakstis, A. J., and Kidd, K. K. (1996) Hum. Genet. 98, 91-101; Ding, Y.-C., Wooding, S., Harpending, H. C., Chi, H.-C., Li, H.-P., Fu, Y.-X., Pang, J.-F., Yao, Y.-G., Yu, J.-G. X., Moyzis, R., and Zhang, Y.-P. (2000) Proc. Natl. Acad. Sci. USA. 97, 14003-14006).

FIG. 2 Nucleotide and amino acid sequences of VNTR motifs. The nucleotide (SEQ. ID NO. 1) and corresponding amino acid (red) sequences of 35 DRD4 exon 3 48 bp repeat motifs are shown. Prior nomenclature (Lichter, J. B., Barr, C. L., Kennedy, J. L., Van Tol, H. H. M., Kidd, K. K. and Livak, K. J. (1993) Human Molecular Genetics 2, 767-773) for 19 of these motifs are indicated (α through ξ). The putative single step origin of most of these motifs is indicated, either as a recombination event (R) or a mutation event (M). For example, the seven motif is hypothesized to be a recombination between a 2 motif and a 3 motif (R 2/3) and the 8 motif is hypothesized to be a single point mutation of a 2 motif (M 2). Motifs 1 though 6, which account for the vast majority of observed haplotype variants (Table 1), are considered the progenitors. Motifs with no putative origin noted (for example, motif 15), have multiple possible progenitors.

FIG. 3 Proposed origin of DRD4 diversity. A simplified model for exon 3 48 bp repeat sequence diversity is shown, with only major recombination events indicated (FIG. 2). The major 2R, 4R, and 7R-alleles are shown in yellow, and the minor 3R, 5R, and 6R-alleles in gray, along with their hypothesized origins by unequal recombination (red arrows). Large red arrows indicate the putative multistep origin of the 7R-allele. Adjacent promoter region (L₁/S₁), exon 1 (L₂ /S₂), and intron 3 (G-G/A-C) polymorphisms are indicated. The strong linkage of the L₁, L₂ and A-C polymorphisms with the DRD4 7R-allele is noted.

FIG. 4. Proposed origin of ADHD/DRD4 allele diversity. A model for DRD4 exon 3 repeat sequence diversity is shown. The ancestral 4R(1-2-3-4) and 7R(1-2-6-5-2-5-4) alleles are noted in yellow, with large red arrows indicating the multistep origin of the 7R-allele. The proposed mutational or recombinational origins of the 12 novel alleles reported in this study are indicated along the blue arrows. Amino acid changes are also indicated. Haplotype nomenclature as described in FIG. 2.

FIG. 5. A simplified diagram of complex genetic disorders. The left colored circles represent the potentially overlapping phenotypes classified together as a single disorder. In the current study, the refined phenotype of ADHD without comorbidity is proposed to represent one of the circles. The Gene 1—Gene N displayed along the DNA molecule indicates our inability to estimate the number of genes associated with the disorder. Likewise, the double-headed arrows represent our inability to predict how these genes interact to produce the phenotype(s) depicted at left. Some fraction of the disorder may have a nongenetic cause (arbitrarily represented as 0.2 nongenetic in the diagram), for example brain damage in the case of ADHD (Swanson, J M, Oosterlaan, J, Murias, M, Schuck, S, Flodman, P, Spence, M A, et al. Attention deficit/hyperactivity disorder children with a 7-repeat allele of the dopamine receptor D4 gene have extreme behavior but normal performance on critical neuropsychological tests of attention. Proc Natl Acad Sci USA 2000; 97, 4754-4759.) The Genes 1—Gene N account for some fraction of the disorder (arbitrarily represented as 0.2 each in the diagram). Two widely discussed models for how genetic variants predispose to common disorders are shown, the Common Variant-Common Disorder (CVCD) hypothesis, and the Allelic Heterogeneity or Rare Variant-Common Disorder (RVCD) hypothesis.

FIG. 6. Contrast between rare single gene disorders and common complex genetic disorders. For single gene disorders, for example Huntington Disease (Huntington's Disease Collaborative Research Group. A novel gene containing a trinucleotide repeat that is unstable on Huntington's Disease chromosomes. Cell 1993; 72, 971-983)(left), predisposing alleles (indicated by a=0.0001) and the disease frequency (indicated by a/x=0.0002) are rare. Therefore, one observes a dramatic increase in allele frequency (and relative risk) in probands. For complex disorders related to common alleles, however, only modest increases in allele frequency (and relative risk) are expected. In the example shown (right), three predisposing alleles (DRD4 7R,b,c) in three different genes are hypothesized to interact. Each allele is proposed to be at polymorphic frequency in the population (0.05-0.12). Individuals with predisposing genotypes [(DRD4 7R/x)(b/x), (DRD4 7R/x)(c/x), (b/x)(c/x)] represent 0.05 of the population, the approximate frequency of ADHD (Faraone, S V, Doyle, A E, Mick, E, and Biederman, J. Meta-analysis of the association between the 7-repeat allele of the dopamine D4 receptor gene and attention deficit hyperactivity disorder. Am J Psychiatry 2001; 158, 1052-1057). The observed increase in alleles DRD4 7R,b, and c in probands ranges from 4-fold (if all cases are caused by these genes) to 2-fold (if only 50% of cases are caused by these genes). For example, a significant fraction of ADHD may have nongenetic causes, yet these cases will be included in our proband population (FIG. 5).

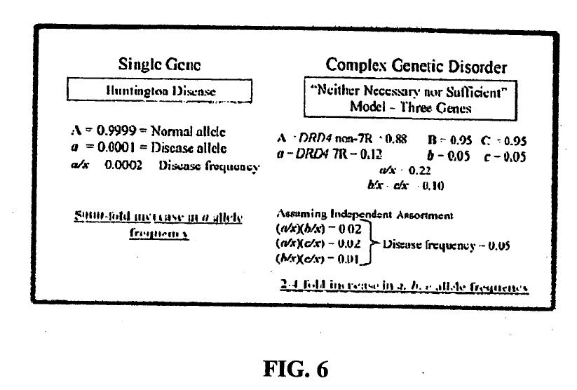

FIG. 7. Polymorphism distribution at the DRD4 locus. Seventy DRD4 polymorphisms are displayed using VG (Nickerson, D. A., Taylor, S. L., Weiss, K. M., Clark, A. G., Hutchinson, R. G., Steingard, J., et al. (1998) Nature Genet 19, 233-240), with individual variants aligned along the horizontal axis. Approximate locations of the variants along the DRD4 loci (GenBank AC021663) are indicated by blue lines reaching to the diagrammatic representation of the gene (above). In this representation, exon positions are represented by blocks (yellow, noncoding, orange, coding; +1=translation start), and the positions of Alu repetitive sequences by pointed blue blocks. The position of a 120 bp upstream duplication and the exon 3 48 bp VNTR are indicated by green triangles. A 288 bp site (−809 To −521) at the promoter region that contains an anomalously high number of SNPs is indicated. These SNPs exhibit little 4R versus 7R frequency difference. Individuals (vertical axis) are grouped by VNTR length (4R/4R, 7R/7R, and 2R/2R) and geographic origin (African, European, etc.) as indicated. Homozygotes for the allele with the highest relative frequency (common allele) are indicated by blue squares, homozygotes for alternative (rare) alleles by yellow squares, and heterozygotes by red squares. The 7R/7R and 2R/2R individuals were greatly oversampled in comparison to their population frequency, and hence common and rare alleles were defined by the frequency in a randomly sampled population.

FIG. 8. Pairwise linkage disequilibrium (D′) at the DRD4 locus. The program GOLD (Abecasis, G. R., and Cookson, W. O. (2000) Bioinformatics 16, 182-183) was used to generate and display all pairwise values of LD for 31 DRD4 polymorphisms with minor alleles >0.01. Separate calculations were performed on 4R/4R (top) and 7R/7R (bottom) populations. The color scale indicated grades LD values from 0.00 (blue) to 1.00 (red). At the short distances used in this study (<6 kb), LD values of approximately 0.6 are expected by chance (Kruglyak, L. (1999) Nature Genet 22, 139-144).

FIG. 9. SNP recombination fraction for DRD4 7R alleles. The observed percent recombination at the 18 SNPs from Table 5 is plotted versus distance from the 7R VNTR. The curve is an empirically determined least squared fit to the data. The diagrammatic representation of the DRD4 locus is as described in FIG. 7.

FIG. 10. A diagrammatic model for DRD4 variant selection. DRD4 2R,4R and 7R protein variants are shown aligned along a scale of relative efficiency for Camp reduction (normalized to 4R=1.0), calculated from the data of Asghari et al (Asghari, V., Sanyal, S., Buchwaldt, S., Paterson, A., Jovanovic, V., and Von Tol, H. H. M. (1995) J. Neurochem. 65, 1157-1165). The diagrammatic protein models were constructed using the rhodopsin crystal structure as a framework. The unusual derivation of the 7R allele from the ancestral 4R allele (approximately 42,500 years ago), and it's increase in prevalence is indicated by a red to blue arrows. The subsequent derivation of the 2R allele from a 7R/4R recombination is indicated by multiple yellow arrows.

### DETAILED DESCRIPTION OF THE INVENTION

All publications mentioned herein are incorporated herein by reference in their entireties. The publications discussed above, below and throughout the text are provided solely for their disclosure prior to the filing date of the present application. Nothing herein is to be construed as an admission that the inventor is not entitled to antedate such disclosure by virtue of prior invention.

As discussed below in more detail, the present inventors have shown that a strong linkage disequilibrium (LD) exists between the 7R-allele of DRD4, disproportionately represented in individuals diagnosed with ADHD, and surrounding DRD4 polymorphisms. Markers within this large LD block thus are useful in the diagnosis of ADHD. It should be noted that due to the strong LD discovered by the present inventors, any marker within this region is potentially useful for diagnosing ADHD, as will be appreciated by one of skill in the art. Such new markers may be identified by techniques well known in the art. Accordingly, the diagnostic reagents of the present invention are not limited to specific DRD4 polymorphisms, but also include other markers now known or subsequently identified in the block of LD surrounding the 7R-allele of DRD4.

Evidence of Positive Selection Acting at the Human Dopamine Receptor D4 Gene Locus

Associations have been reported of the 7-repeat (7R) allele of the human dopamine receptor D4 (DRD4) gene with both attention deficit/hyperactivity disorder (ADHD) and the personality trait of novelty seeking. This polymorphism occurs in a 48 bp tandem repeat (VNTR) in the coding region of DRD4, with the most common allele containing four repeats (4R), and rarer variants containing two to eleven. Here, we show by DNA resequencing/haplotyping of 600 DRD4 alleles, representing a worldwide population sample, that the origin of 2R- through 6R-alleles can be explained by simple one-step recombination/mutation events. In contrast, the 7R-allele is not simply related to the other common alleles, differing by greater than 6 recombinations/mutations. Strong linkage disequilibrium (LD) was found between the 7R-allele and surrounding DRD4 polymorphisms, suggesting this allele is at least 5-10 fold “younger” than the common 4R-allele. Based on an observed bias towards nonsynonymous amino acid changes, the unusual DNA sequence organization, and the strong LD surrounding the DRD4 7R-allele, we propose that this allele originated as a rare mutational event that nevertheless increased to high frequency in human populations by positive selection.

The human DRD4 gene (Van Tol, H. H. M., Bunzow, J. R., Guan, H.-C., Sunahara, R. K., Seeman, P., Niznik, H. B. and Civelli, O. (1991) Nature 350, 610-614), located near the telomere of chromosome 11p, is one of the most variable human genes known. Most of this diversity is the result of length and single nucleotide polymorphism (cSNP) variation in a 48 bp tandem repeat (VNTR) in exon 3, encoding the third intracellular loop of this dopamine receptor. Variant alleles containing two (2R) to eleven (11R) repeats are found, with the resulting proteins having 32 to 176 amino acids at this position. Interestingly, the frequency of these alleles varies widely. The 7R-allele, for example, has an extremely low incidence in Asian populations, yet a high frequency in the Americas.

A number of investigations have found associations between particular alleles of this highly variable gene and behavioral phenotypes (La Hoste, G. J., Swanson, J. M., Wigal, S. B., Glabe, C., Wigal, T., King, N., and Kennedy, J. L. (1996) Molecular Psychiatry 1, 21-24; Swanson, J. M., Flodman, P, Kennedy, J, Spence, M. A., Moyzis, R, Schuck, S, Murias, M, Moriarity, J, Barr, C, Smith, M, et al., (2000) Neuroscience and Behavioral Reviews 24, 21-25; Swanson, J. M., et al. (2000) Proc. Natl. Acad. Sci. USA 97, 4754-4759; Ebstein, R. P., Novick, O., Umansky, R., Priel, B., Osher, Y., Blaine, D., Bennett, E. R., Nemanov, L., Katz, M., and Belmaker, R. H. (1996) Nature Genetics 12, 78-80; Benjamin, J., Li, L., Patterson, C., Greenberg, B. D., Murphy, D. L. and Hamer, D. H. (1996) Nature Genetics 12, 81-84). While initial studies suggested that the 7R-allele of the DRD4 gene might be associated with the personality trait of novelty seeking, the most reproduced association is between the 7R-allele and attention deficit/hyperactivity disorder (ADHD) (Swanson, J., Deutsch, C., Cantwell, D., Posner, M., Kennedy, J., Barr, C., Moyzis, R., Schuck, S., Flodman, P., and Spence, M. A. (2001) Clinical Neuroscience Research 1, 207-216). ADHD is the most prevalent disorder of early childhood, affecting an estimated 3% of elementary school children. As defined by DSM-IV criteria (Am. Psychiatr. Assoc. (1994) DSM-IV: Diagnostic and Statistical Manual of Mental Disorder (Forth Edition) (Am. Psychiatr. Assoc., Washington, D.C.)), ADHD consists of developmentally inappropriate inattention, impulsivity and hyperactivity with early onset (before the age of 7). Evidence of a strong genetic component of ADHD has come from a variety of twin, adoption, and family studies (Faraone, S. V. and Biederman, J. (1994) Child Adolesc. Clin. North Am. 3, 285-291; Thaper, A., Holmes, J., Poulton, K., and Harrington, R. (1999) Br. J. Psychiatry 174, 105-111). The efficacy of methylphenidate in the treatment of ADHD indicated that genes in the dopamine pathway might play a role in the syndrome's etiology (Volkow, N. D., Wang G. J., Fowler, J. S., Fischman, M., Foltin, R., Abumrad, N. N., Gatley, S. J., Logan, J, Wong, C, Gifford, A, et al., (1999) Life Sci. 65, 7-12). Initial association studies found ADHD probands to exhibit an increased frequency of DRD4 7R-alleles in comparison to controls. Eight separate replications of this initial observation have now been reported. As in all association studies, however, one can not assume that the presence of a DRD4 7R-allele is either necessary or sufficient to “cause” ADHD. Further work will be required to understand the genetic/environmental factors underlying this behavior.

Nevertheless, given the likely functional importance of this change in the DRD4 protein, in a region that couples to G-proteins and mediates post-synaptic effects (Asghari, V., Sanyal, S., Buchwaldt, S., Paterson, A., Jovanovic, V., and Von Tol, H. H. M. (1995) J. Neurochem. 65, 1157-1165), these association studies have generated considerable interest. In particular, this association is consistent with the common variant-common disorder (CVCD) hypothesis, which proposes that the high frequency of many complex genetic diseases is related to common DNA variants (Collins, F. S., Guyer, M. S., and Chakravarti, A. (1997) Science 278, 1580-1581; Zwick, M. E., Cutler, D. J., and Chakravarti, A. (2000) Ann. Rev. Genomics Hum. Genet. 1, 387-407). However, many questions remain as to the nature of the DRD4 /ADHD association. One would like to know 1) if particular 7R-allele variants are associated with ADHD, 2) the population distribution of variant DRD4 alleles, and/or 3) whether the observed marker is in linkage disequilibrium (LID) with other etiologically relevant polymorphisms. Given the known high level of sequence polymorphism of this gene, PCR-based DNA resequencing is the most efficient and accurate method to address these questions. Here, we use this approach to determine A) the population distribution of DRD4 exon 3 haplotypes and B) their relative association with adjacent polymorphisms. We present haplotype data indicating that the DRD4 7R-allele originated as a rare mutational event (or events), that nevertheless increased to high frequency in human populations by positive selection.

Methods

Population Samples. Samples were obtained as reported previously (Chang, F.-M., Kidd, J. R., Livak, K. J., Pakstis, A. J., and Kidd, K. K. (1996) Hum. Genet. 98, 91-101; Ding, Y.-C., Wooding, S., Harpending, H. C., Chi, H.-C., Li, H.-P., Fu, Y.-X., Pang, J.-F., Yao, Y.-G., Yu, J.-G. X., Moyzis, R., and Zhang, Y.-P. (2000) Proc. Natl. Acad. Sci. USA. 97, 14003-14006). The origins of the 600 alleles reported in this study, based on geographical/ethnic origin, are as follows: North and South America, 12.7% (76 alleles), Europe, 36.7% (220 alleles), Asia, 27.3% (164 alleles), Africa, 20.3% (122 alleles), and Pacific, 3.0% (18 alleles). Lymphoblastoid cell lines have been established for most of these population samples, and methods for transformation, cell culture, and DNA purification described. For LD studies of the DRD4 4R-G-G SNP association, an additional 288 alleles (approximately equally derived from African, Asian and European sources) were used. All persons gave their informed consent prior to their inclusion in this study, carried out under protocols approved by the Human Subjects Committees at the participating institutions.

PCR Amplification and DNA sequencing. PCR amplification of the DRD4 promoter polymorphism was conducted as described (Seaman, M. I., Fisher, J. B., Chang, F.-M., and Kidd, K. D. (1999) Am. J. Med. Genet. 88, 705-709; McCracken, J. T., Smalley, S. L., McGough, J. J., Crawford, L., Del'Homme, M., Cantor, R. M., Liu, A., and Nelson, S. F. (2000) Mol. Psychiatry 5, 531-536). The program OLIGO 6.0 was used to select primer pairs for the exon 1 polymorphism (Catalano, M., Nobile, M., Novelli, E., Nothen, M. M., and Smeraldi, E. (1993) Biol. Psychiat. 34, 459-464) (5′-TGGGCCGCCGCATTCGT-3′ (SEQ. ID NO. 2) and 5′-GGTGGGTGTATCGCCGAGGGA-3′ (SEQ. ID. NO. 3); 661-nucleotide product) and the exon 3 VNTR (5′-CGTACTGTGCGGCCTCAACGA-3′ (SEQ. ID NO. 4) and 5′-GACACAGCGCCTGCGTGATGT-3′ (SEQ. ID NO. 5); 705 nucleotide product for the 4R-allele). For some amplifications of the VNTR, primers described previously were used (Lichter, J. B., Barr, C. L., Kennedy, J. L., Van Tol, H. H. M., Kidd, K. K. and Livak, K. J. (1993) Human Molecular Genetics 2, 767-773). The alternative primers were chosen farther from the VNTR, to minimize out-of-register hybridization during amplification. PCR reactions were conducted in 25 microliter volumes, containing 100ng genomic DNA, 200 micromolar dXTPs, 0.5 micromole of each primer, 1X PCR buffer (Qiagen), 1X Q-solution (Qiagen) and 0.625 units Taq DNA polymerase (Qiagen). Amplification was performed using Perkin-Elmer 9700 thermal cyclers. A 20 second, 96-degrees C. hot start was used, followed by 40 cycles of 95 degrees C. for 20 seconds and 68 degrees C. for 1 minute. Following a 4-minute chase at 72-degrees C., excess primers were eliminated with 0.5 units of Shrimp Alkaline Phosphatase (SAP, Amersham Life Science), 0.1 unit of Exonuclease I (Exo I, Amersham Life Science) and 1X SAP buffer (Amersham Life Science). The SAP/Exo I reaction was carried out at 37 degrees C. for 1 hour, followed by a 15-minute heat inactivation at 72-degrees C. The DNA from the SAP/Exo I reaction was used directly for DNA sequencing. For most individuals, the two allelic PCR products were first separated on 1.2-% agarose gels. DNA cycle sequencing was conducted by standard techniques, using ABI 377 and 3700 automated sequencers (Riethman, H. C., Xiang, Z., Paul, S., Morse, E., Hu, X.-L., Flint, J., Chi, H.-C., Grady, D. L., and Moyzis, R. K. (2001) Nature 409, 948-951). DNA sequences of the DRD4 haplotypes reported herein have been submitted to GenBank (Accession numbers AF395210 through AF395264).

Kₐ/Kₛ and Allele age calculations. Kₐ/Kₛ ratios were calculated by standard methods (Kimura, M. (1968) Nature 217, 624-626; Kreitman, M. (2000) Ann. Rev. Genomics Hum. Genet. 1, 539-559). Putative recombinant haplotypes were not considered independent events. Allele age calculations were conducted by standard methods (Harpending, H. and Rogers, A. (2000) Annu. Rev. Genomics Hum. Genet. 1, 361-385; Kimura, M. and Ohta, T. (1973) Genetics 75, 199-212; Slatkin, M. and Rannala, B. (2000) Ann. Rev. Genomics Hum. Genet. 1, 225-249; Serre, J. L., Simon-Bouy, B., Mornet, E., Jaume-Roig, B., Balassopoulou, A., Schwartz, M, Taillandier A, Boue J, Boue A., (1990) Hum. Genet. 84, 449-454). Briefly:

1) Calculated from Population Frequency.

E (t₁)=[−2p/(1−p)] In (p), where E(t₁)=expected age, time is measured in units of 2N generations, and p=population frequency. For DRD4, p=19.2% for the 7R-allele and 65.1% for the 4R-allele. A generation time of 20-25 years and N=10,000 were assumed (regarded as a minimum estimate of the effective population size of modern humans during the period prior to recent growth).

2) Calculated from Intra-Allelic Variation.

t=[1/ln(1−c)] ln [(x(t)−y)/(1−y)], where t=allele age, c=recombination rate, x(t)=frequency in generation t, and y=frequency on normal chromosomes. Assuming the origin of the 7R-allele was on a L₁L₂(7R)A-C haplotype, for the (7R)A-C association c=0.0000136 (from the average recombination rate per Mb times the VNTR-SNP distance), x(t)=97% (the percent of A-C SNPs associated with DRD4 7R-alleles), and y=13.9% (the percent of A-C SNPs associated with African DRD4 4R-alleles, assumed to be the “normal” allele). For the promoter polymorphism L₁(7R) association, c=0.000165, x(t)=90.8%, and y=61.9%.

Results and Discussion

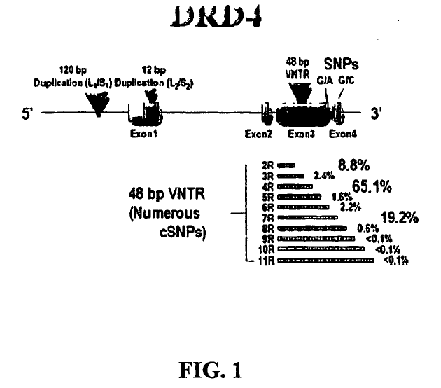

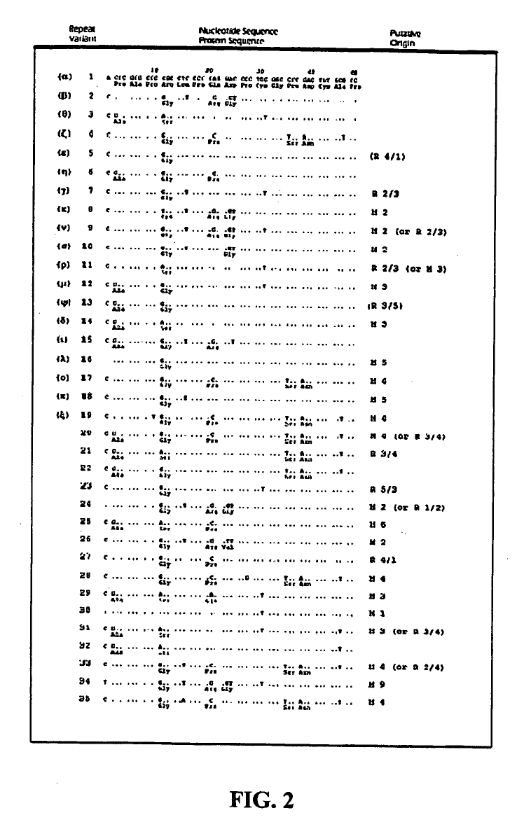

Primer sets were chosen to amplify the four exons of the highly GC-rich DRD4 gene, as well as the adjacent promoter region and splice junctions (FIG. 1). Initial resequencing of the entire promoter and coding region of the DRD4 gene from 20 ADHD probands (data not shown) uncovered a number of polymorphisms reported previously. These polymorphisms included two insertion/deletion polymorphisms, one in the promoter region (4.3 kb upstream of the VNTR) and one in exon 1 (2.7 kb upstream of the VNTR; see FIG. 1). In addition, a number of new coding SNPs were uncovered in the exon 3 48 bp VNTR, as well as two previously unreported SNPs in intron 3, 20 nucleotides apart and approximately 350 bp downstream from the center of the VNTR (FIG. 1). Given the high level of VNTR polymorphism identified in this initial sample, a more extensive PCR-resequencing of 600 exon 3 VNTR alleles was conducted, obtained from a worldwide population sample (Table 1 and FIG. 2). This sample contained individuals representing most major geographical origins (see Methods). The majority of individuals were heterozygotes, and the two allelic PCR products could be separated by gel electrophoresis prior to sequencing, providing unambiguous haplotypes. Altogether, we screened over 450,000 bp of genomic DNA and 2,968 48 bp repeats.

TABLE 1
Haplotypes of 600 DRD4 exon 3 alleles

| Allele | F | N | Haplotype |
|---|---|---|---|
| 2R | 0.088 | 55 | |
| | | 43 | 1-4 |
| | | 12 | 30-4* |
| 3R | 0.024 | 36 | |
| | | 16 | 1-7-4 |
| | | 9 | 1-2-4 |
| | | 4 | 1-11-33* |
| | | 3 | 1-9-4 |
| | | 1 | 1-2-22 |
| | | 1 | 1-2-21 |
| | | 1 | 1-2-31 |
| | | 1 | 1-2-32 |
| 4R | 0.651 | 250 | |
| | | 238 | 1-2-3-4 |
| | | 3 | 1-2-14-4 |
| | | 2 | 1-2-13-4 |
| | | 2 | 1-2-12-4 |
| | | 1 | 1-17-3-4 |
| | | 1 | 1-9-12-4 |
| | | 1 | 1-8-3-4 |
| | | 1 | 1-10-3-4 |
| | | 1 | 1-9-3-4 |
| 5R | 0.016 | 27 | |
| | | 12 | 1-3-2-3-4* |
| | | 4 | 1-2-13-34-4* |
| | | 3 | 1-2-2-3-4 |
| | | 2 | 1-2-6-5-4 |
| | | 2 | 1-11-2-3-4 |
| | | 1 | 1-3-2-14-4 |
| | | 1 | 1-2-6-23-4 |
| | | 1 | 1-2-3-9-4 |
| | | 1 | 1-2-3-27-4 |
| 6R | 0.022 | 24 | |
| | | 16 | 1-2-3-2-3-4 |
| | | 2 | 1-2-6-5-2-20 |
| | | 2 | 1-2-6-5-2-4 |
| | | 1 | 1-2-14-17-2-4 |
| | | 1 | 1-6-5-2-5-4 |
| | | 1 | 1-2-13-2-5-19 |
| | | 1 | 24-6-5-2-5-4 |
| 7R | 0.192 | 199 | |
| | | 177 | 1-2-6-5-2-5-4 |
| | | 5 | 1-2-6-5-2-5-19* |
| | | 3 | 1-2-6-5-2-3-4 |
| | | 3 | 1-2-6-5-13-5-4* |
| | | 2 | 1-8-25-5-2-5-4 |
| | | 2 | 1-2-3-5-2-5-4 |
| | | 1 | 1-2-6-5-2-13-4 |
| | | 1 | 1-2-29-17-2-5-4 |
| | | 1 | 1-2-6-2-2-5-4 |
| | | 1 | 1-8-25-5-2-3-4 |
| | | 1 | 1-2-6-16-2-3-4 |
| | | 1 | 1-2-6-5-2-14-4 |
| | | 1 | 1-2-3-17-2-5-4 |
| 8R | 0.006 | 6 | |
| | | 2 | 1-2-6-5-17-2-13-35* |
| | | 1 | 1-2-6-5-2-2-5-4 |
| | | 1 | 1-2-6-26-5-26-3-35 |
| | | 1 | 1-2-6-26-5-26-3-4 |
| | | 1 | 1-2-6-18-5-18-3-4 |
| 9R | <0.001 | 1 | 1-8-25-5-2-5-2-23-4 |
| 10R | <0.001 | 1 | 1-2-15-6-2-6-5-2-5-4 |
| 11R | <0.001 | 1 | 1-2-3-27-5-23-25-5-2-5-28 |

F, observed allele frequency in 2,836 chromosomes from 37 worldwide human populations (3, 17);
N, allele number identified by sequence analysis in this study (non-4R alleles were oversampled by 2-3-fold); haplotype, haplotypes are indicated using the repeat motif nomenclature proposed (FIG. 2). Alleles with adjacent asterisks indicate common variants found only in a single population sample (2R 30-4, [symbol] ; 3R 1-11-33, [symbol] ; 5R 1-3-2-3-4, Chinese; 5R 1-2-13-34-4, [symbol] ; 7R 1-2-6-5-2-5-19, [symbol] ; 7R 1-2-6-5-13-5-4, [symbol] ; 8R 1-2-6-5-17-2-13-35, [symbol] Alleles with a single representation by definition were found in only one population

In the 600 chromosomes sequenced, 56 different haplotypes were found (Table 1). These haplotypes were composed of 35 distinct 48-bp variant motifs (FIG. 2), 19 of which were reported previously (designated Alpha through Xi in FIG. 2). We propose that these DRD4 48 bp variant motifs are given numbers as shown, rather than the letters used previously, since there are not enough characters in the Greek alphabet. We propose that DRD4 exon 3 variants be designated in the format shown, i.e., the most common 4R allele being designated 4R(1-2-3-4), etc.

We intentionally over sampled non-4R-alleles approximately two-fold, since little sequence variation was uncovered in the common 4R-allele (Table 1), even though it represents 65 percent of the world population frequency. Most of the haplotypes in this sample (85.7%) were found at frequencies less than 1% (Table 1). Looking at nucleotide diversity among variants defined by their VNTR number, the common 2R, 4R, and 7R-alleles exhibit the least diversity, with 78.2%, 95.2%, and 88.9% of the alleles respectively represented by the most common 2R(1-4), 4R(1-2-3-4), and 7R(1-2-6-5-2-5-4) haplotypes (Table 1). In contrast, while the 3R, 5R, 6R, and 8R alleles are rarer, they have proportionally more variants (Table 1). This unusual pattern of allele diversity is clearly not a simple length effect, i.e., longer alleles have greater diversity. Many population specific rare haplotypes were observed. Examples include the 2R(30-4) haplotype found only in the Surui (South America) sample, and the 5R(1-3-2-3-4) haplotype found only in the Han Chinese (Asian) sample (Table land FIG. 2).

The pattern of nucleotide variation observed in the VNTR haplotypes is not random (FIG. 2). Most DNA sequence variants change the amino acid sequence, sometimes quite dramatically (i.e., Gln to Pro; FIG. 2). Although many of these variants are related mutational events (below), one can account for these relationships in calculating Kₐ/Kₛ (the ratio of the number of amino acid replacements per site divided by the estimate of the number of synonymous changes). Values of Kₐ/Kₛ greater than 1 are usually taken to be a stringent indicator of positive selection at the observed DNA segment. For a tandem repeat sequence, many assumed relationships can be inferred, and hence different Kₐ/Kₛ ratios calculated. For all assumed relationships of the DRD4 variants, however, Kₐ/Kₛ>1. For example, assuming that the most abundant 1 through 6-variant motifs (FIG. 2) all have a common origin, and that diversity was generated by both mutation and recombination (below), a Kₐ/Kₛ value of 3 is obtained. Expanding this analysis to include between-species divergence (a powerful method to improve these calculations) is not possible, due to the rapid de novo generation of variation in this VNTR in primate lineages (Livak, K. J., Rogers, J., and Lichter, J. B. (1995) Proc. Natl. Acad. Sci. USA 92, 427-431).

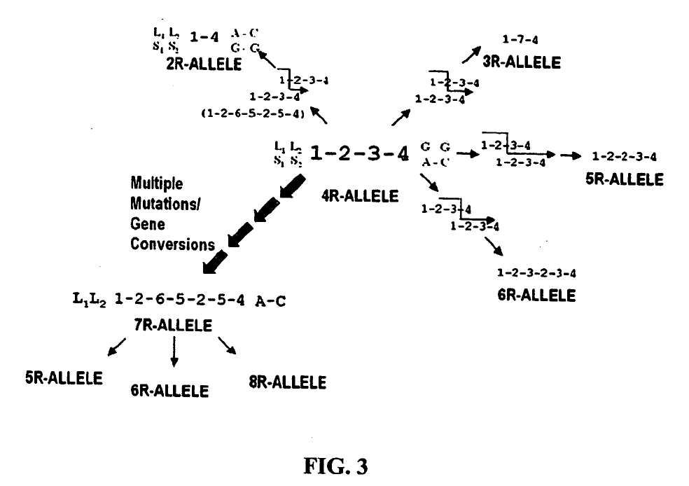

Standard approaches to defining evolutionary relationships between these haplotypes are not applicable, due to the repetitive nature of the DNA sequence. Based on the observed DNA sequences and their nucleotide variations, however, it is straightforward to propose a simple origin for the majority of these haplotypes (FIG. 3; Table 1). One-step recombination/mutation events between the most common alleles can account for nearly all of the observed variation of the 2R through 6R alleles. FIG. 3 is a simplified diagram of the most common recombination events proposed. While the inferred nucleotide sequence of an ancestral DRD4 cannot be determined, all alleles in a particular primate species appear to be derived from a relatively recent common ancestor. The most prevalent 4R-allele is proposed as the human progenitor allele, based on 1) limited sequence data reported for primate DRD4 4R-alleles, 2) the lower level of LD for polymorphisms surrounding this allele (as discussed below), and 3) the sequence motif arrangements of the non-4R alleles. Unequal recombination between two 4R(1-2-3-4) alleles would produce the observed common 2R though 6R alleles (FIG. 3). The position of crossover determines the resulting sequence. For example the most common 3R(1-7-4) and 3R(1-2-4) alleles differ only in the position of crossover, either within or after the second repeat (FIG. 3; Table 1). Thus, the known high frequency of unequal recombination between tandem repeats (Jeffreys, A. J., Neil, D. L., and Neumann, R. (1998) EMBO J. 17, 4147-4157) can account for most of the observed diversity of the DRD4 gene.

In addition to unequal crossovers, single point mutations are evident in this population sample (Table 1 and FIG. 2). For example, with one exception, all 2R alleles worldwide have the sequence 2R(1-4) (Table 1). All twelve 2R alleles resequenced from Surui (South American) DNA were found to contain a single point mutation, the 2R(30-4) allele (Table 1 and FIG. 2). This mutation, a C to T change in the first repeat, does not alter the amino acid sequence, and likely has a recent (less than 10,000-20,000 year) origin.

In contrast, the formation of the observed 7R and higher alleles cannot be explained by simple one-step recombination/mutation events from the 4R(1-2-3-4) haplotype (FIG. 3). The generation of a 7R allele from the most prevalent 4R allele would require at least one recombination and 6 mutations to arise. Even allowing for more complicated gene conversion events, multiple low probability steps are needed to convert a 4R allele into a 7R allele (FIG. 3). For example, the central 5-variant motif found in the common 7R(1-2-6-5-2-5-4) haplotype could be produced by a recombination between two 4R-alleles. Recombination between the terminal 4-variant motif of one 4R-allele and the initial 1-variant motif of the second 4R-allele would yield a 7R(1-2-3-5-2-3-4) haplotype (FIG. 2). Three additional mutations of each of the two three-variant motifs in this putative 7R-haplotype are then required to produce the current 7R(1-2-6-5-2-5-4) haplotype. Four of these six nucleotide changes are nonsynonymous, altering the amino acid sequence (Ser to Gly, Gln to Pro, Ala to Pro, and Ser to Gly; FIG. 2). While gene conversion rather than mutation could be proposed as the mechanism to “insert” these nucleotide changes in a hypothetical 7R(1-2-3-5-2-3-4) allele, two unlikely events, one involving 7R-7R allele gene conversion, would be necessary (FIGS. 2 and 3).

None of these putative “intermediate” 7R haplotypes were observed in this worldwide population sample. Our sample included 47 7R-alleles sequenced from individuals of African origin, thought to contain populations with the greatest genetic diversity and age. It is unlikely, then, that “intermediate” 7R haplotypes exist at high frequency. It is not our intention, however, to propose a specific origin of the DRD4 7R-allele. Rather, we wish to emphasize that, based on DNA sequence analysis, the DRD4 7R-allele appears to be quite distinct from the common 2R through 6R alleles. It is impossible to determine if the origin of the DRD4 7R-allele was a single, highly unlikely event, or a series of unlikely events (FIG. 3).

Regardless of the mechanism of origin of the DRD4 7R-allele, it is clearly capable of participating in recombination events with the other alleles. Most of the rare 7R haplotypes observed appear to be recombination events, mostly with the common 4R(1-2-3-4) allele (Table 1). For example, the 7R(1-2-6-5-2-3-4) haplotype appears to be a recombination between a 4R(1-2-3-4) allele and a 7R(1-2-6-5-2-5-4) allele (Table 1 and FIG. 2). This origin was confirmed by analyzing SNPs outside the recombination region (see below). Further, the origin of some of the rare 5R and 6R alleles and all of the 8R and higher alleles can be explained by recombinations involving a 7R allele, since they contain the 6-variant motif, unique to the 7R allele (FIG. 2 and Table 1). Many of these 8R and higher alleles, however, appear to have more complicated origins, based on DNA sequence analysis (Table 1 and FIG. 2).

This model (FIG. 3) explains the apparent anomaly in the observed haplotype diversity noted above (Table 1), where the most abundant (and ancient, see below) 4R-allele has the lowest nucleotide diversity. If recombination is the predominant generator of diversity, then the majority of 4R/4R recombination events are predicted to have unchanged nucleotide sequence. Such events can only be inferred by recombination of outside markers. Only when out-of-register recombination occurs will new nucleotide sequence (and length) variants be generated (FIG. 3). The observed pattern of haplotype diversity is consistent with a predominantly “2-allele” system (4R and 7R), with most of the rarer variants generated by recombination from these two haplotypes (FIG. 3).

The unusual nature of the sequence organization of the DRD4 7R-allele, suggesting it arose as a rare mutational event, led us to determine if differences in LD exist between the 4R and 7R-alleles. The haplotype of two adjacent intronic SNPs (G/A-G/C; FIG. 1) could be directly determined, since they were present on the same PCR product used to amplify the 48 bpVNTR. Strong LD was found between the A-C SNP pair and the 7R-allele (FIG. 3). Ninety-seven percent of 7R-alleles were associated with the A-C SNP pair (66 out of 68 examined). The two 7R alleles associated with G-G SNPs were 7R/4R recombinant haplotypes, as determined originally from DNA sequence analysis (above). In contrast, both the G-G and A-C SNP pairs are associated with DRD4 4R-alleles (487 examined alleles). However, the G-G pair is most frequent, representing 86.1% of the African sample, but up to 98.6% of our Asian sample.

All African 7R-alleles were associated with the A-C haplotypes, while only 13.9% of African 4R-alleles were associated with the A-C haplotype. DNA sequence analysis of several chimp and bonobo samples (data not shown) indicates that the G-G SNP pair is likely the ancestral sequence (FIG. 3). Thus, it appears that the original DRD4 7R allele arose on this rarer A-C SNP background. A sample of 73 2R, 3R, 5R, and 6R-alleles showed approximately equal association with the G-G and A-C SNPs, consistent with their proposed recombinational origin from both the 4R and 7R-alleles (FIG. 3). Interestingly, all 26 Asian 2R-allele samples examined showed association with the A-C SNPs, suggesting their origin from recombinations involving 7R-alleles (FIG. 3).

Similar results were obtained for more distant promoter and exon 1 insertion/deletion polymorphisms (FIG. 1). In this case association was inferred indirectly from data obtained for our prior population studies and PCR analysis of a subset of the individuals used in this study. For forty samples where parental DNA was also available and could be genotyped for these markers, phase could be directly inferred. Strong association was observed between the long (duplicated) L₁ promoter polymorphism (FIG. 1) and the 7R-allele (FIG. 3), with 90.8% of 7R-alleles associated with L₁ (607 alleles analyzed). In contrast, the L₁ polymorphism is coupled with only 61.9% of 4R-alleles (2102 alleles analyzed). While population specific variation was observed (for example, more L₁-4R coupling in Chinese than African populations), little overall L₁-4R linkage was detected (FIG. 3). The closer L₂ polymorphism in exon 1 (FIG. 1) was associated with 93.4% of 7R-alleles and 86.4% of 4R-alleles, a relative difference similar to that observed for the L₁-7R and L₁-4R association. The L₂/S₂ polymorphism is in a coding region, however, and selective constraints may be influencing allele frequency as well (Seaman, M. I., Chang, F.-M., Deinard, A. S., Quinones, A. T., and Kidd, K. K. (2000) J. Exp. Zool. 288, 32-38).

Standard methods of estimating coalescence time for these alleles are not applicable, given the repetitive nature of the region and the high recombination frequency. However, calculations of allele age based on the relatively high worldwide population frequency of the DRD4 4R and 7R-alleles suggest that these alleles are ancient (>300,000 years old) (see Methods). On the other hand, calculations of allele age based on the observed intra-allelic variability (see Methods) suggest the 7R-allele is 5-10 fold “younger” (30,000-50,000 years old). Such large discrepancies between allele ages calculated by these two methods are usually taken as evidence that selection has increased the frequency of the allele to higher levels than expected by random genetic drift. The absolute values of these estimates are greatly affected by the assumptions used in their computations, for example the assumed recombination frequency. We have used conservative estimates of recombination frequency, based on the average observed for the terminal 20 Mb of 11p (International Human Genome Sequencing Consortium (2001) Nature 409, 860-921). Given the observed high recombination at this locus (Table 1 and FIG. 3), it is likely that the actual age of the 7R-allele is even younger, and further LD analysis will refine these estimates. The important conclusion, however, is that regardless of the parameters assumed, the relative age differences for the 4R and 7R-alleles calculated from intra-allelic variability remains large, while their population frequency suggests they are both ancient.

The simplest hypothesis to account for 1) the observed bias in nucleotide changes (Kₐ/Kₛ), 2) the unusual sequence organization of the DRD4 7R-allele, and 3) the strong LD surrounding this allele, is that the 7R-allele arose as a rare mutational event (or events), that nevertheless increased to high frequency by positive selection. Advantageous alleles usually take a long time to reach a frequency of 0.1, then increase rapidly to high frequencies (>0.9). While it is possible we are observing the recent expansion of a highly advantageous 7R-allele, it is more likely, we suggest, that this “two-allele” DRD4 system (FIG. 3) is an example of balanced selection. Such selection may be more pervasive in the human genome than generally thought. A balanced selection model proposes that both the 4R and 7R-alleles are maintained at high frequencies in human populations. A variety of mechanisms could be proposed for such balanced selection, ranging from heterozygote advantage to frequency-dependent selection. According to evolutionary game theory (Smith, J. M. Evolution and the Theory of Games. (1982) Cambridge: Cambridge University Press), the evolutionary payoff for a particular kind of personality will depend on the existing distribution of personality types. For example, high aggression may lead to high fitness if almost everyone is meek, but might result in low fitness when very common, as aggressive individuals would suffer the penalties of frequent conflict. This type of frequency-dependent selection might be expected to apply to many types of psychological variation, including those associated with this particular neurotransmitter receptor.

Alternative explanations to the proposed positive selection, such as recent random bottlenecks, population expansion, and/or population admixture are less likely to account for the observed results. Bottlenecks have certainly occurred during human migration and evolution (Tishkoff, S. A., Dietzsch, E., Speed, W., Pakstis, A. J., Kidd, J. R., Cheung, K., Bonne-Tamir, B., Santachiara-Benerecetti, A. S., Moral, P., and Krings, M. (1996) Science 271, 1380-1387; Chen, C., Burton, M., Greenberger, E., and Dmitrieva, J. (1999) Evol. Hum. Behav. 20, 309-324; Reich, D. E., Cargill, M., Bolk, S., Ireland, J., Sabeti, P.C., Richter, D. J., Layery, T., Kouyoumjian, R., Farhadian, S. F., Ward, R., and Lander, E. S. (2001) Nature 411, 199-204), and have undoubtedly influenced the current worldwide DRD4 allele frequency. Numerous population studies on other genes have shown that an “Out of Africa” constriction of allele diversity (and an increase in LD) likely occurred. In the present study, a greater diversity (and lower LD) was found for African DRD4 4R-alleles in comparison to the remainder of our population sample, which is consistent with the “Out of Africa” hypothesis. While one could argue that the 7R-allele frequency was increased by chance during the Out-of-Africa expansion, this does not explain the unusual lack of diversity in African 7R-alleles. The most common L₁L₂-7R(1-2-6-5-2-5-4)-A-C haplotype (FIG. 3) is found at frequencies comparable to those found worldwide (>85%). It is difficult to imagine what type of bottleneck could produce such results, i.e., strong worldwide LD for a single allele (DRD4 7R) yet little LD for the remaining alleles. A model that is consistent with the observed results is the “weak Garden of Eden” (wGOE) hypothesis, where the DRD4 4R-allele would be hypothesized to be ancient and present in indigenous populations, while the 7R-allele was spread by the expansion out of (and into) Africa. In such a wGOE hypothesis, positive selection for the DRD4 7R-allele must still be proposed.

Although we suggest that a recent mutational origin and positive selection best account for the DRD4 7R-allele data, another possibility can not be ruled out. Given the highly unlikely recombination/mutation events required to generate the 7R-allele from the 4R-allele, a possibility worth considering is the importation of this allele from a closely related hominid lineage. What lineage that might be can only be speculated, but Neanderthal populations were present at the approximate time the 7R-allele originated. Under this model, the coalescence time for the 4R and 7R-alleles would then be ancient, with the importation occurring only recently, as measured by LD. Obviously, additional experimental work may clarify these speculations.

For the DRD4 locus, it is unlikely that selection for an adjacent gene can account for the proposed selection, given the distinct and unusual DNA sequence of the DRD4 7R-allele itself. If the DRD4 7R-allele originated roughly 40,000 years ago, one might ask what was occurring at that time in human history? It is tempting to speculate that the major expansion of humans that occurred at that time, the appearance of radical new technology (the upper Paleolithic) and/or the development of agriculture, could be related to the increase in DRD4 7R-allele frequency. Perhaps individuals with personality traits such as novelty seeking, perseverance, etc. drove the expansion (and partial replacement)? The speculation that migration could account for the current 7R-allele distribution has been proposed. In addition to such phenotypic selection, sexual selection could be operating as well. As originally defined by Darwin (Darwin, C. The Descent of Man and Selection in Relation to Sex. (1874) New York: Merrill and Baker), “any advantage which certain individuals have over others of the same sex and species solely in respect of reproduction” will lead to increased offspring. If individuals with a DRD4 7R-allele have personality/cognitive traits that give them an advantage (multiple sexual partners, higher probability for mate selection, etc.) then the frequency of this allele will expand rapidly, depending on the cultural milieu. Perhaps cultural differences can account for some of the observed differences in DRD4 7R-allele frequency. Obviously, determining the exact nature of the DRD4 selection, and its biochemical and behavioral basis, awaits further experimentation. Recent experiments, indicating that individuals with ADHD and possessing this unusual DRD4 7R-allele perform normally on critical neuropsychological tests of attention in comparison to other ADHD probands, point to but one of many areas of future investigation.

One may ask why an allele that appears to have undergone strong positive selection in human populations nevertheless is now disproportionately represented in individuals diagnosed with ADHD? The CVCD hypothesis proposes that common genetic variation is related to common disease, either because the disease is a product of a new environment (so that genotypes associated with the disorder were not eliminated in the past) or the disorder has small effect on fitness (because it is late onset). For early onset disorders (such as autism, ADHD, etc.) we suggest entertaining the possibility that predisposing alleles are in fact under positive selection, and only result in deleterious effects when combined with other environmental/genetic factors. In this context, it is possible that prior selective constraints are no longer operating on this gene. It is also possible to speculate, however, that the very traits that may be selected for in individuals possessing a DRD4 7R-allele may predispose behaviors that are deemed inappropriate in the typical classroom setting, and hence diagnosed as ADHD.

### High Prevalence of Rare Dopamine Receptor D4 (DRD4) Alleles in Children Diagnosed with Attention Deficit Hyperactivity Disorder ADHD)

Associations have been reported of the 7-repeat (7R) allele of the human dopamine receptor D4 (DRD4) gene with both the personality trait of novelty seeking and attention deficit/hyperactivity disorder (ADHD). The increased prevalence of the 7R-allele in ADHD probands is consistent with the common variant-common disorder (CVCD) hypothesis, which proposes that the high frequency of many complex genetic disorders is related to common DNA variants. Based on the unusual DNA sequence organization and strong linkage disequilibrium surrounding the DRD4 7R-allele, we proposed above that this allele originated as a rare mutational event, that nevertheless increased to high prevalence in human populations by positive selection (see also, Ding et. al., Proc. Natl. Acad. Sci. USA 99, 309-314, 2002). We have now determined, by DNA resequencing of 250 DRD4 alleles obtained from 132 ADHD probands, that most ADHD 7R-alleles are of the conserved haplotype found in our previous 600 allele worldwide DNA sample. Interestingly, however, half of the 24 haplotypes uncovered in ADHD probands were novel (not one of the 56 haplotypes found in our prior population studies). Over 10 percent of the ADHD probands had- these novel haplotypes, most of which were 7R-allele derived. The probability that this high incidence of novel alleles occurred by chance in our ADHD sample is much less that 0.0001. These results suggest that allelic heterogeneity at the DRD4 locus may also contribute to the observed association with ADHD.

Attention Deficit Hyperactivity Disorder (ADHD) is a neurobehavioral disorder defined by symptoms of developmentally inappropriate inattention, impulsivity, and hyperactivity with early onset. Current estimates indicate that 3-6% of school age children are diagnosed with ADHD, making it the most prevalent disorder of childhood. While the broad DSM-IV phenotype of ADHD almost certainly has multiple biological etiologies, numerous family, twin and adoption studies have documented a strong genetic basis (Faraone, S V, Biederman, J. Genetics of attention-deficit hyperactivity disorder. Child Adolesc Clin North Am 1994; 3, 285-291). However, given high cross-national variation in the recognition and treatment of ADHD, we proposed that the ADHD Combined type (DSM-IV) without serious comorbidity should be used as a “refined” phenotype in biological and genetic research (Swanson, J M, Sergeant, J A, Taylor, E, Sonuga-Barke, E J S, Jensen, and Cantwell, D P. Attention deficit disorder and hyperkinetic disorder. Lancet 1998; 351, 429-433).

Despite the high heritability of ADHD, initial genome scan studies have failed to identify genes of major effect, although a region on chromosome 16p13 has been implicated in subsequent studies by the same group. Such negative results are not unexpected for a complex genetic disorder like ADHD, where phenotypic heterogeneity is likely, and the practical but (to date) restricted sample sizes limit statistical power. Candidate gene studies, on the other hand, require much smaller sample sizes to achieve the same statistical power. The efficacy of a dopamine agonist drug, methylphenidate, in the treatment of ADHD has suggested that genes in the dopamine pathway may be involved in the disorder's etiology. This dopamine hypothesis of ADHD suggests a number of candidate genes that could logically be tested for their association with the disorder. The draft human genome sequence has provided information sufficient to examine multiple candidate genes in parallel, often representing most of the proteins in a relevant biochemical pathway.

One of these candidate genes, DRD4, located near the telomere of chromosome 11p, is one of the most variable human genes known. Most of this diversity is the result of length and single nucleotide polymorphism (cSNP) variation in a 48 bp tandem repeat (VNTR) in exon 3, encoding the third intracellular loop of this dopamine receptor. Variant alleles containing two (2R) to eleven (11R) repeats are found, with the resulting proteins having 32 to 176 amino acids at this position.

A number of investigations have found associations between particular alleles of this highly variable gene and behavioral phenotypes. While some studies have suggested that the 7R-allele of the DRD4 gene might be associated with the personality trait of novelty seeking (Klugar, A N, Siegfried, Z, and Ebstein, R P. A meta-analysis of the association between DRD4 polymorphism and novelty seeking. Mol Psychiatry 2002; 7, 712-717), the most reproduced association is between the 7R-allele and attention deficit/hyperactivity disorder (ADHD). Above, we showed by DNA resequencing/haplotyping of 600 DRD4 alleles, representing a worldwide population sample, that the origin of 2R- through 6R-alleles can be explained by simple one-step recombination/mutation events. In contrast, the 7R-allele is not simply related to the other common alleles, differing by greater than 6 recombinations/mutations. Strong linkage disequilibrium (LD) was found between the 7R-allele and surrounding DRD4 polymorphisms, suggesting this allele is at least 5-10 fold “younger” than the common 4R-allele. Based on an observed bias towards nonsynonymous amino acid changes, the unusual DNA sequence organization, and the strong LD surrounding the DRD4 7R-allele, we proposed that this allele originated as a rare mutational event, that nevertheless increased to high frequency in human populations by positive selection.

Why is the DRD4 7R allele, which arose recently and underwent strong positive selection, nevertheless now disproportionately represented in individuals diagnosed with ADHD? We suggested that selection for an adjacent polymorphism was unlikely, given the distinct and unusual DNA sequence organization of the DRD4 7R allele itself. The DRD4 7R allele is at moderate prevalence in most populations that have been examined for ADHD (approximately 10-15%). Therefore, the approximate two-fold increase in DRD4 7R allele frequency in ADHD probands (λ=1.9), calculated from a recent meta-analysis is consistent with the Common Variant-Common Disorder (CVCD) hypothesis (also called the Common Disease-Common Variant hypothesis) (Reich, D E, Lander, E S. On the allelic spectrum of human disease. Trends in Genetics 2001; 17, 502-510). In the CVCD hypothesis, the high prevalence of a given disorder (and its associated alleles) is attributed to either 1) the interaction with a new environment (such that genotypes associated with the disorder were not eliminated in the past) or 2) the disorder has small effect on fitness (because it is late onset). We suggest a third possibility. Perhaps predisposing alleles in fact are under positive selection, and only result in deleterious effects when combined with other environmental/genetic factors. This would explain the high prevalence of common disorders in the population, since the selected allele would only be deleterious in a small fraction of those individuals carrying it. Positive selection for particular human alleles may, in fact, be common (Harpending, H and Rogers, A. Genetic perspectives on human origins and differentiation. Ann Rev Genomics Hum Genet 2000; 1, 361-385; Tishkoff, SA, Varkonyl, R, Cahinhinan, N, Abbes, S, Argyropoulos, G, Destro-Bisol, G, et al. Haplotype diversity and linkage disequilibrium at human G6PD: recent origin of alleles that confer malarial resistance. Science 2001; 293, 455-462), contributing to the observation of unexpectedly large blocks of LD in the human genome (Reich, D E, Cargill, M, Bolk, S, Ireland, J, Sabeti, PC, Richter, DJ, et al. Linkage disequilibrium in the human genome. Nature 2001; 411, 199-204; Daly, M J, Rioux, J D, Schaffner, S F, Hudson, T J, and Lander, E S. High-resolution haplotype structure in the human genome. Nature Genetics 2001; 29, 229-232; Patil, N, Berno, A J, Hinds, D A, Barrett, W A, Doshi, J M, Hacker, C R, et al. Blocks of limited haplotype diversity reveled by high-resolution scanning of human chromosome 21. Science 2001; 294, 1719-1723; Sabeti, P C, Reich, D E, Higgins, J M, Levine, H Z P, Richter, D J, Schaffner, S F, et al. Detecting recent positive selection in the human genome from haplotype structure. Nature 2002; 419, 832-837).

It is a reasonable hypothesis that high prevalence human genetic disorders will be related to some common variants in the population. However, it is unclear that single common variants will be the only relevant variants. Alleles at low prevalence, most of which have not be identified by current SNP searches targeting a small sample size (The International SNP Map Working Group. A map of human genome sequence variation containing 1.42 million single nucleotide polymorphisms. Nature 2002; 409, 928-933), could also contribute to complex disease (Pritchard, J K. Are rare variants responsible for susceptibility to complex diseases? Am J Hum Genet 2001; 69, 124-137). Of the hundreds of “single hit” disease genes identified to date, the vast majority contain hundreds of “private” mutations that alter protein function. In order to test this Rare Variant-Common Disorder (RVCD) model for complex disease, much greater depth of DNA resequencing must be conducted, ideally in individuals enriched for the putative mutant alleles (i.e., probands).

All previous studies of the DRD4/ADHD association have defined alleles based only on PCR length differences. Hence, it is possible that specific sequence variants are actually associated with the disorder. For example, one could imagine that the selected DRD4 7R allele might have a higher mutation rate than the common 4R allele, and it is in fact these variant 7R alleles that predispose to ADHD. Given the large sequence diversity of this gene, in which 56 different exon 3 haplotypes were uncovered in 600 chromosomes obtained from a worldwide sample, we decided that direct DNA resequencing of DNA obtained from ADHD probands was the only method that could answer this question.

Here, we confirm the increased prevalence of DRD4 7R alleles in individuals diagnosed with the refined phenotype of ADHD (La Hoste, G J, Swanson, J M, Wigal, S B, Glabe, C, Wigal, T, King, N and Kennedy, J L. Dopamine D4 receptor gene polymorphism is associated with attention deficit hyperactivity disorder. Mol Psychiatry 1996; 1, 21-24). By DNA resequencing of 250 DRD4 alleles obtained from 132 ADHD probands, we show that most ADHD associated 7R-alleles are of the conserved haplotype found in our previous 600 allele worldwide DNA sample. Interestingly, however, over 10 percent of the ADHD probands had novel DRD4 haplotypes, not previously found in our worldwide allele sample. The probability that this high prevalence of novel alleles occurred by chance in our ADHD sample is much less that 0.0001. Most of these novel haplotypes were 7R-allele derived. These results suggest that allelic heterogeneity (the RVCD model) may also be contributing to the association of the DRD4 locus with ADHD, as is routinely found for “single-gene” genetic disorders.

Materials and Methods

Clinical. ADHD probands were recruited to participate in either clinical trials or the Multimodality Treatment Study of Children with ADHD (MTA; MTA Cooperative Group. A 14-month randomized clinical trial of treatment strategies for attention deficit/hyperactivity disorder. Arch Gen Psychiatry 1999; 56, 1073-1086) at the University of California, Irvine. The refined phenotype of ADHD was diagnosed by a research assessment battery described in detail elsewhere (Hinshaw, S P, March, J S, Abikoff, H, Arnold, L E, Cantwell, D P, Conners, C K, et al. Comprehensive assessment of childhood attention-deficit hyperactivity disorder in the context of a multisite, multimodel clinical trial. J Attention Disorders 1997; 1, 217-234), that includes psychiatric interviews and questionnaires about the symptoms of the disorder and other psychopathological behavior related to comorbid disorders. Instruments used included the Diagnostic Interview Schedule for Children, Fourth Version (DISC-IV), the SNAP-IV Rating Scale and a locally developed family and developmental history questionnaire. In addition, measures of ability and achievement were obtained using the Wechsler Intelligence Scale for Children, Third Revision (WISC-III) and the Wechsler Individual Achievement Test (WIAT). The inclusion criteria included a DSM-IV diagnosis of ADHD-Combined Type, which requires the endorsement of at least six of the nine symptoms of inattention and six of the nine symptoms of hyperactivity/impulsivity. High cutoffs on parent and teacher ratings of ADHD items on the SNAP rating were required. Subjects with an IQ score on the WISC-III<80 were excluded. Information was also obtained for oppositional defiant disorder (ODD), but a comorbid diagnosis of ODD did not exclude the subject. A diagnosis of other comorbid disorders (such as Tourette Syndrome), or treatment of symptoms of other disorders with non-stimulant psychotropic drugs, were exclusion criteria for this study.

Establishing Cell Lines and DNA Purification. Lymphoblastoid cell lines were established for all ADHD probands. Methods for transformation, cell culture, and DNA purification have been described above (see also, Chang, F-M, Kidd, J R, Livak, K J, Pakstis, A J, and Kidd, K K. The world-wide distribution of allele frequencies at the human dopamine D4 receptor locus. Hum Genetics 1996; 98, 91-101).

PCR amplification and DNA sequencing. The DRD4 exon 3 VNTR was amplified with primer sets described previously (5′-CGTACTGTGCGGCCTCAACGA-3′(SEQ. ID NO. 4) and 5′-GACACAGCGCCTGCGTGATGT-3′ (SEQ. ID NO. 5); 705 nucleotide product for the 4R-allele). PCR reactions were conducted in 25 microliter volumes, containing 10 ng genomic DNA, 200 micromolar dXTPs, 0.5 micromole of each primer, 1×PCR buffer (Qiagen), 1X Q-solution (Qiagen) and 0.625 units Taq DNA polymerase (Qiagen). Amplification was performed using Perkin-Elmer 9700 thermal cyclers. A 20 second, 96-degrees C. hot start was used, followed by 40 cycles of 95 degrees C. for 20 seconds and 68 degrees C. for 1 minute. Following a 4-minute chase at 72-degrees C., excess primers were eliminated with 0.5 units of Shrimp Alkaline Phosphatase (SAP, Amersham Life Science), 0.1 unit of Exonuclease I (Exo I, Amersham Life Science) and 1×SAP buffer (Amersham Life Science). The SAP/Exo I reaction was carried out at 37 degrees C. for 1 hour, followed by a 15-minute heat inactivation at 72-degrees C. The DNA from the SAP/Exo I reaction was used directly for DNA sequencing. For individuals heterozygous for DRD4 alleles, the two allelic PCR products were first separated on 1.2-% agarose gels. DNA cycle sequencing was conducted by standard techniques, using ABI 3100 and 3700 automated sequencers. Overall PCR/resequencing success was greater than 95%. One allele from an ADHD proband, 9R(1-8-25-5-2-5-2-23-4), was included in our prior worldwide sample. DNA sequences of the novel DRD4 haplotypes reported herein have been submitted to GenBank (Accession numbers AY151027-AY151038).

Analysis of sequence data: Analysis of sequence data was accomplished using PHRED, PHRAP, POLYPHRED and CONSED (Ewing, B, Hiller, L, Wendl, M C, and Green, P. Base-calling of automated sequencer traces using phred. I. Accuracy assessment. Genome Res 1998; 8, 175-185; Ewing, B and Green, P. Base-calling of automated sequencer traces using phred II. Error probabilities. Genome Res 1998; 8, 186-194; Nickerson, D A, Tobe, V O, and Taylor, S L. Polyphred: automating the detection and genotyping of single nucleotide substitutions using fluorescence-based resequencing. Nucleic Acids Res 1997; 14, 2745-2751; Nickerson, D A, Taylor, S L, Weiss, K M, Clark, A G, Hutchinson, R G, Stengard, J, et al. DNA sequence diversity in a 9.7-kb region of the human lipoprotein lipase gene. Nature Genetics 1998; 19, 233-240). These programs are used to clean and assemble the sequence files, and aid in the detection of DNA polymorphism. For every position in the DRD4 consensus sequence, POLYPHRED examines each sample sequence for evidence of polymorphism/heterozygosity. The rank limit for identifying a position as polymorphic is under user control. Based on our experience, we have configured POLYPHRED to identify all potential polymorphisms of rank 1-4, which are then independently evaluated by two skilled investigators.

Capture of individual genotypes/haplotypes into a database (SNPMAN). The collection of SNPs into a relational database is done via an in-house software package we have designated SNPMAN. SNPMAN is a package of 3 main programs written originally in PERL and SQL and now available in both binaries and open source format. The first program (SNPMAN) is designed to collect the SNP information from POLYPHRED output files and transform it into acceptable SQL command files, later to be executed by a database operator (DBO). The second program (MANIP) is the CONSED-addon extension that allows an experienced chromatogram reader to adjust or delete database information in case of false positive or false negative polymorphisms. The last program (GIMMEPRETTYBASE) in the SNPMAN package converts existing polymorphism tables into acceptable input files for visual genotyping via VG.

Statistical Analysis. Allele distributions were compared using Fischer's Exact test for a 2×k table, as implemented in SAS (v.6.12, running on a SUN Ultra2 Enterprise workstation). In our prior worldwide sample (above), all DRD4 repeat lengths except for 4R were oversampled by a factor of two. This was corrected for before comparisons were conducted with the present sample.

Results

DNA was isolated from 132 probands diagnosed with the refined phenotype of ADHD, sequentially identified as part of ongoing research and clinical trials programs at the UCI Child Development Center (see Methods). Table 2 gives the demographics, ADHD symptoms and psychometric test scores of these probands. As expected, the majority (80%) of individuals were of European ancestry and male. On the SNAP, average rating per item summary scores of inattention and hyperactivity/impulsivity above 2.0 are considered severe. The average SNAP for this group of probands was 2.22 (Table 2). Ratings were also obtained for oppositional defiant disorder (ODD), often found to be comorbid with ADHD. The observed average for ODD (1.62) was significantly higher than for population norms. Other psychometric measures of IQ (WISC) and achievement (WIAT) were in the normal range for the group (Table 2).

TABLE 2
Demographics, ADHD symptoms, and psychometric
test scores of the ADHD probands.

| | Females | Males | Total | |
|---|---|---|---|---|
| | (26) | (106) | (132) | SD |
| Demographics | | | | |
| Age | 8.62 | 8.83 | 8.78 | 1.88 |
| % European | | | 79.7 | |
| % Hispanic | | | 11.4 | |
| % African American | | | 3.6 | |
| % Asian | | | 2.8 | |
| % Native American | | | 2 | |
| % Pacific Island | | | 0.4 | |
| ADHD symptoms | | | | |
| Inattention average | 2.53 | 2.3 | 2.41 | 0.52 |
| Hyperactivity/impulsivity | 2.07 | 1.96 | 2.01 | 0.74 |
| ADHD average | 2.29 | 2.15 | 2.22 | 0.51 |
| ODD average | 1.68 | 1.57 | 1.62 | 0.81 |
| Psychometric tasks | | | | |
| WISC block design | 101.9 | 113.3 | 107.7 | 33.9 |
| WISC vocabulary | 96.9 | 105.4 | 101.2 | 30.4 |
| IQ average | 99.4 | 109.4 | 104.4 | 25.7 |
| WIAT reading | 96.5 | 100.5 | 98.5 | 14.6 |
| WIAT math | 100.6 | 101.4 | 101.9 | 13.8 |
| WIAT spelling | 96.6 | 97.8 | 97.2 | 13.8 |
| Achievment average | 97.8 | 99.9 | 98.9 | 12.5 |

The exon 3 VNTR region of the DRD4 gene was amplified from these DNAs, and the distribution of DRD4 genotypes obtained in this sample is shown in Table 3. As reported in numerous other studies, including our own, the frequency of ADHD individuals with at least one DRD4 7R allele is approximately two-fold greater (43.2%) than found in ethnically matched control individuals. Interestingly, the observed frequency of 2R and 3R alleles was also increased in this ADHD sample (Table 3). In European populations the observed allele frequency for DRD4 is 2R=0.07, 3R=0.03, 4R=0.73, 5R=0.01, 6R=0.02, 7R=0.12, 8R<0.01, 9R<0.001 (2N=1652; ref. 20 and 22 and unpublished data). Calculating an expected genotype distribution (assuming Hardy-Weinberg equilibrium, Table 3) indicates that only 22% of Europeans should have a 7R/x geneotype, consistent with prior experimental control data from our research. Adjusting these values for the increased frequency of 7R alleles in some non-European populations can not account for the increased frequency in our predominantly European ancestry ADHD sample (Table 2).

TABLE 3
Genotypes of 132 ADHD Probands.

| | Genotype | | | | | | | | | | | |
|---|---|---|---|---|---|---|---|---|---|---|---|---|
| | 2R/4R | 3R/3R | 3R/4R | 4R/4R | 4R/6R | 2R/7R | 3R/7R | 4R/7R | 6R/7R | 7R/7R | 4R/8R | 4R/9R |
| Observed | 20(19) | 1 | 9(8) | 43(41) | 2(1) | 5 | 3 | 42 | 1 | 4 | 1 | 1 |
| Expected | 14 | <1 | 6 | 70 | 4 | 2 | 1 | 23 | <1 | 2 | 1 | <1 |

DNA sequence analysis of 250 DRD4 alleles obtained from these ADHD probands found 24 different haplotypes (Table 4). No data were obtained on 14 alleles (5.3%; two 2R, seven 4R and five 7R alleles) due to PCR and/or sequencing failures. Altogether, we screened over 200,000 bp of genomic DNA and 1,132 48-bp repeats. Interestingly, only half (12/24) of the observed haplotypes (Table 4) were identified previously in our analysis of 600 DRD4 alleles obtained from a worldwide population sample (see GenBank accession nos. AF395210-AF395264). For example, using our proposed nomenclature for DRD4 haplotypes (FIG. 2), the majority of 7R alleles found in our ADHD probands (45/55 =81.8%) are the common 7R(1-2-6-5-2-5-4) haplotype (Table 4). In this nomenclature, the numbers in brackets refer to different 48 bp repetitive sequence motifs (FIG. 2). Likewise, the majority of 2R and 4R alleles were the common 2R(1-4) and 4R(1-2-3-4) haplotypes, respectively. These three common alleles (2R,4R, and 7R) account for 87.2% of the observed alleles (Table 4), similar to the proportion obtained in our 600 allele population sample. The remaining 9 alleles are rare 3R,4R,6R, and 7R variants observed previously (Table 4).

TABLE 4
Haplotypes of 250 DRD4 exon3 alleles
from 132 ADHD probands.

| Allele | N | Haplotype |
|---|---|---|
| 2R | 23 | 1-4 |
| 3R | 14 | |
| | 8 | 1-7-4 |
| | 3 | 1-9-4 |
| | 2 | 1-2-20 |
| | 1 | 1-6-4 |
| 4R | 156 | |
| | 150 | 1-2-3-4 |
| | 2 | 1-2-14-4 |
| | 2 | 1-2-5-4 |
| | 1 | 1-2-6-4 |
| | 1 | 1-26-3-4 |
| 6R | 3 | |
| | 1 | 1-2-3-2-3-4 |
| | 1 | 1-2-6-5-2-20 |
| | 1 | 1-2-6-2-5-4 |
| 7R | 55 | |
| | 45 | 1-2-6-5-2-5-4 |
| | 2 | 1-2-6-5-2-5-19 |
| | 2 | 1-2-6-1-2-3-4 |
| | 1 | 1-2-6-5-2-23-4 |
| | 1 | 1-2-6-5-8-5-4 |
| | 1 | 1-2-14-5-2-5-4 |
| | 1 | 1-2-3-17-2-5-4 |
| | 1 | 1-8-25-5-2-5-4 |
| | 1 | 1-2-6-5-2-3-4 |
| 8R | 1 | 1-2-6-26-5-2-3-4 |
| 9R | 1 | 1-8-25-5-2-5-2-23-4 |

N: allele number identified by sequence analysis; haplotype nomenclature is described in FIG. 1. Alleles in normal font, were identified previously in a survey of 600 world-wide alleles.²² Alleles in bold are unique to this study.

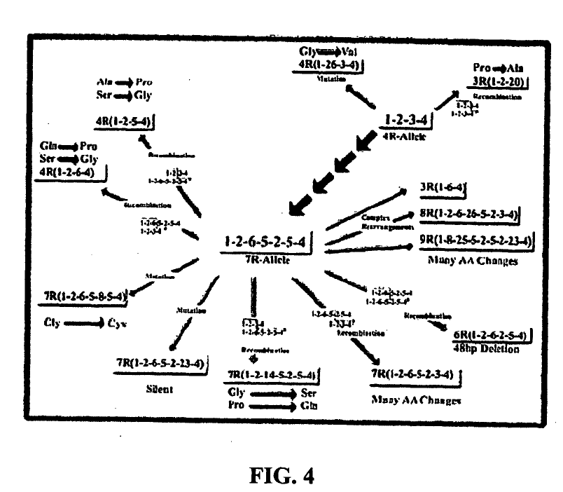

The other half of the observed haplotypes were unique, not identified in our extensive prior analysis (Table 4). Excluding the common variants, expected to be present in all samples, sixty percent (12/20) of rare (<0.01 frequency) variants found in this ADHD sample were unique. Fifteen ADHD probands had one of these 12 unique DRD4 haplotypes (15/132=11.4%). For seven of these probands, parental DNA was available. PCR resequencing indicated that the variant allele was present in one of the parents, and not a new mutation. All but one of these 12 novel alleles produce an altered amino acid sequence in the resulting DRD4 protein compared to the common allele (FIG. 4). For example, the observed 4R(1-2-6-4) variant would substitute a Gly for a Ser and a Pro for a Gln in comparison to the common 4R (1-2-3-4) variant (Table 4). This result is similar to our prior population studies on the DRD4 gene, where 87% of the observed rare variants altered the amino acid sequence of the resulting protein.

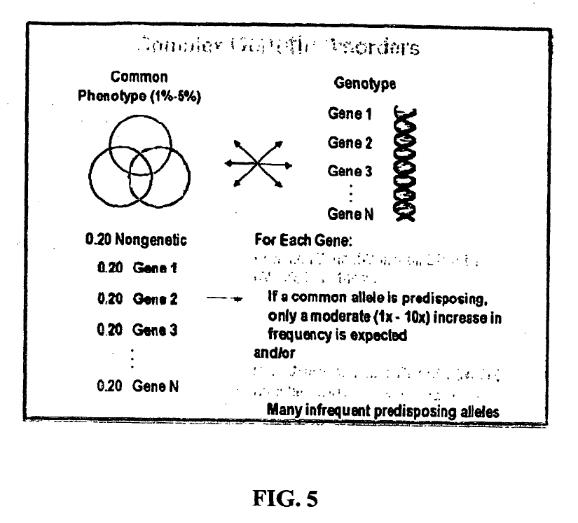

The origin of most of these newly observed variants can be inferred to be 7R allele derivatives, based on their nucleotide sequence (FIGS. 4 and 5). The 5 and 6 variant motifs (FIG. 2) are diagnostic for the 7R allele, found only in this allele and its derivatives. Ten of the 12 haplotype variants contain these motifs (Table 4), and hence likely arose as recombination/mutation events involving a 7R allele (FIG. 4). For example, the 4R(1-2-5-4) allele likely arose as a recombination event between a 4R(1-2-3-4) allele and a 7R(1-2-6-5-2-5-4) allele. Genotyping six of these variant alleles for flanking SNPs diagnostic for the 4R and 7R alleles confirmed their hypothesized origin (data not shown; FIG. 4). The finding that the majority of these rare variants are derived from 7R alleles should be contrasted with our prior population studies of the DRD4 gene, in which rare variants were found to be equally derived from 4R and 7R alleles. There is an approximate two-fold increase in rare 7R alleles in this ADHD sample in comparison to our prior population sample (18.2% versus 11.0%; Table 4).

Including these 7R-related sequence variants in the 7R allele category removes 5 individuals from the non-7R category, originally classified based on their PCR fragment length (numbers in brackets in Table 3). Altogether, individuals with 7R and derivative 7R alleles account for 47% of the ADHD proband population (Table 3).

Twenty percent of the ADHD DRD4 alleles sequenced in this study are of non-European origin (Table 2). However, 33% (5/15) of the individuals with novel rare alleles were non-European in genetic origin. While this difference is not statistically significant, it is possible that population stratification could account for a portion of the observed difference. Our prior worldwide sequence sample included 220 European DRD4 alleles, as well as 164 Asian, 122 African, 76 North and South American, and 18 Pacific Island ancestry alleles. Non-4R alleles were oversampled approximately two-fold in this prior study. Given the ethnic breakdown of our ADHD probands (Table 2), then, our prior worldwide resequencing sample can serve as an extensively “oversampled” control, in which we have comparable numbers of European origin alleles, and 10-20 fold larger numbers of non-European alleles.

Sixty-seven different haplotype variants of DRD4 were seen in either our prior population sample, our ADHD sample (Table 4), or both. Sixty of these haplotypes are at low (<0.01) frequency. We can therefore ask a simple question: How likely is it, assuming a pool of uncommon DRD4 alleles, that these two samples (population control and ADHD) would give the observed results? Most of the rare alleles were found only once, hence we can only estimate their frequency in the population. Our initial sample size of 600 chromosomes, however, is expected to detect eighty percent of variants at a frequency of 0.002 or greater. Based on DRD4 allele frequency distributions (Table 4 and above), where the six common 2R-7R alleles account for >90% of the observed alleles, we can estimate that there can be, at most, 85 different DRD4 alleles at frequencies greater than 0.001. At a minimum, therefore, we have identified 79% (67/85) of DRD4 alleles with a frequency greater than 0.001. Alleles less frequent than 0.001 would be found rarely in population samples of the current size, and hence can not contribute significantly to the observed distributions.

One can consider the possibility, then, that among a pool of uncommon alleles, there were 12 undetected alleles (on 15 chromosomes) that happened by chance to occur among the 250 chromosomes obtained from ADHD individuals. Likewise, one can consider the possibility that these 12 alleles were not found among 600 random chromosomes. We considered a range of allele frequencies for these 12 alleles, from 1/400 each to 1/1000 each. For each set of allele frequencies, the probability of seeing none of these 12 new alleles among the 600 chromosomes examined previously can be easily calculated as a multinomial probability (Probability “A”). Likewise, the probability of seeing 9 of these new alleles once, and 3 twice, among 250 chromosomes can be calculated (Probability “B”). For all sets of allele frequencies, either probability “A” or probability “B” is much less than 0.0001. It is extremely unlikely that the distribution of alleles in these two samples has occurred by chance.

We also considered the possibility that this difference is related not to the diagnosis of ADHD, but rather to population stratification. Indeed, one of the reasons we sequenced such a large worldwide sample was to address this issue. We constructed a series of comparison groups from our worldwide population sample. Each comparison group contained the 220 alleles from samples of European origin. Added to this was a random selection from the remaining non-European samples to approximate the ethnic distribution of the ADHD sample (Table 1). In all cases the allele distribution differed significantly between the ADHD sample and the comparison group (p<<0.0001). It is extremely unlikely, therefore, that population stratification and undetected ethnic bias can account for the distribution differences in our population and ADHD samples. We conclude, then, that the most likely reason for the observed differences was our ascertainment of this sample by diagnosis of ADHD, and that variants present at low frequency in the general population were “enriched” in the ADHD sample.

Discussion

The increased frequency of the DRD4 7R allele in ADHD probands is consistent with the predictions of the CVCD hypothesis (FIG. 5). By DNA resequencing from probands diagnosed with the refined phenotype of ADHD, we determined that the majority (83%) of 7R alleles in these individuals were of the common 7R(1-2-6-5-2-5-4) haplotype found previously (Table 3). However, we uncovered an unusually high prevalence (50%) of novel haplotypes in the 24 haplotypes observed in our sample, most 7R allele derivatives (Table 4). Greater than 10% of ADHD probands had one of these rare alleles. Including these rare derivatives (determined by sequence analysis) in the “7R” class increased the number of ADHD individuals with 7R alleles from 43.2% to 47% (Tables 3 and 4). It is impossible to know without further biochemical/physiological/behavioral experimentation if these derivatives are functionally equivalent/related to 7R alleles (see below). It is likely, however, that all previous studies of the DRD4 /ADHD association modestly underestimated the relative risk by only examining repeat length rather than DNA sequence.

What can account for the high frequency of novel alleles uncovered in the present study? If recombination/mutation were random, one would expect that the majority of derivative alleles would have 4R origins, since this is the most common allele, even in ADHD probands. The DRD4 4R allele is also older than the 7R allele, and hence there has been greater time to accumulate mutations in this allele (unless they have been selected against). In our prior population study, approximately equal numbers of 4R and 7R derivative alleles were uncovered, suggesting a mutation/recombination bias toward 7R alleles (or a stronger selection against 4R variants). In comparison to our prior population survey, however, over 90% of the rare derivative alleles in this ADHD sample have 7R origins (FIG. 4).

We estimate that there are less than 85 DRD4 alleles with population frequency greater than 0.001, and we have identified a minimum of 79% of these alleles. While there could be hundreds of extremely rare DRD4 alleles (at a population frequency of 0.0001), such alleles could only contribute a few examples to our original population sample. Therefore, given the sample sizes used in this and our prior population study, it is expected that, at most, 2-3 alleles might be found only in one sample and not the other. It is extremely unlikely (p<<0.0001), therefore, that finding 12 new alleles (on 15 chromosomes) in the ADHD population was due to chance or population stratification. We propose, then, that our ascertainment of the sample by diagnosis of ADHD was the reason for this observed increase in derivative DRD4 7R alleles.

Further studies, including more extensive population sampling, can refine the number and frequency distribution of rare DRD4 alleles. In particular, it would be informative to know if rare DRD4 alleles exhibit biased geographic/ethnic ancestry distributions. Such information would be essential for the design and interpretation of replicate studies of the current work. In addition, family based analyses can help determine if rare alleles are preferentially transmitted to ADHD probands. However, for behavioral disorders such as ADHD, such studies should be interpreted with caution. It is common in such disorders to be unable to consent key members of a trio (mostly fathers). An inability to ascertain a truly “random” sample of parental genotypes (for example, if there is preferential absence of a parent transmitting a putative predisposing gene) could contribute to biases in tests such as the TdT (West, A, Langley, K, Hamshere, M L, Kent, L, Craddock, N, et al. Evidence to suggest biased phenotypes in children with attention deficit hyperactivity disorder from completely ascertained trios. Mol Psychiatry 2002; 7, 962-966).

The high frequency of amino acid changing variants in these rare haplotypes (>90%), and the low probability that we uncovered these variants by chance (p<<0.0001) suggest that allelic heterogeneity is also playing a role in the association of the DRD4 gene and ADHD (RVCD Model, FIG. 5). The finding of allelic heterogeneity for the DRD4 /ADHD association should not be surprising, since “private” mutations are found frequently for the majority of “single-hit” genetic diseases, even ones where a particular variant is common. For example, while the common AF508 mutation is found in 70% of cystic fibrosis probands, hundreds of rarer mutations have also been identified (Serre, J L, Simon-Bouy, B, Mornet, E, Jaume-Roig, B, Balassopoulou, A, Schwarz, M, et al. Studies of RFLP closely linked to the cystic fibrosis locus throughout Europe lead to new considerations in population genetics. Hum Genet 1990; 84, 449-454). There is no strong experimental or theoretical reason why genes associated with complex genetic disorders involving multiple genes should utilize a different mutational spectrum than genes for single-hit disorders. We suggest, then, that both CVCD association and allelic heterogeneity (RVCD) contribute to the association of the DRD4 gene and ADHD (FIG. 5). The observation of increased allelic heterogeneity adds further support to the hypothesis that the DRD4 gene itself, rather than an adjacent variant in strong LD with DRD4, is responsible for the association.

While data exist indicating that DRD4 protein variants containing different VNTR lengths exhibit different biochemical properties (Asghari, V, Sanyal, S, Buchwaldt, S, Paterson, A, Jovanovic, V, and VanTol, H H M. Modulation of intercellular cyclic AMP levels by different human dopamine D4 receptor variants. J Neurochem 1995; 65, 1157-1165; Jovanovic, V, Guan, H—C, and VanTol, H H M. Comparative pharmacological and functional analysis of the human dopamine D4.2 and D4.10 receptor variants. Pharmacogenetics 1999; 9, 561-568), little is known of the effect of sequence (amino acid) differences in this region of the protein. The functional importance of changes at this position in the DRD4 protein, however, in a region that couples to G proteins and mediates postsynaptic effects (Civelli, O, Bunzow, J R, Grandy, D K. Molecular diversity of the dopamine receptors. Annu Rev Pharmacol Toxicol 1993; 32, 281-307), seems likely. For example, many of the observed changes are quite dramatic [i.e., substituting a Pro for a Gln in 4R(1-2-6-4); FIG. 4], and might be expected to alter the DRD4 protein structure/function. Clearly, further biochemical studies would be helpful. Such studies should be interpreted with caution, however. Observed biochemical differences do not necessarily imply differences at the behavioral level. Many genetic/biochemical systems exhibit great buffering capacity, and biochemical variation often has little physiological effect (Hartman, J L, Garvik, B, and Hartwell, L. Principles for the buffering of genetic variation. Science 2001; 291, 1001-1004). Likewise, not finding biochemical differences between DRD4 variant proteins does not imply that functional differences do not exist at a behavioral level. It is often unclear which biochemical parameter is relevant to test, especially for proteins like DRD4, where most of the interacting proteins are as yet unknown. Further, subtle biochemical changes, difficult to detect in vitro and in vivo, can have large effects at the organismal level. The decade long search for the relevant biochemical basis of Huntington Disease, following the identification of the mutation, is but one recent example. For these reasons, we suggest that genetic approaches will remain more powerful than biochemical approaches at detecting associations with behavioral disorders. We therefore suggest that in addition to further biochemical analysis of DRD4 variants, direct genotype/phenotype correlations continue to be pursed, including brain imaging and model organism experiments (Dulawa, S C, Grandy, D K, Low, M J, Paulas, M P, and Geyer, M A. Dopamine D4 receptor-knock-out mice exhibit reduced exploration of novel stimuli. J Neurosci 1999; 19, 9550-9556). It is the physiological/behavioral outcome of genetic variation that is most relevant. The finding that individuals with ADHD who possess a DRD4 7R allele perform normally on critical neuropsychological tests of attention in comparison to other ADHD probands points to but one of many areas of future investigation.

Based on the current work and the hypothesized origin of human DRD4 diversity, we suggest that future studies might group individuals based on DRD4 genotype differently then in the past. Only VNTR length was considered, usually split into 7R(+) and 7R(−) categories. The DRD4 locus appears to behave like a “two-allele” system (4R and 7R) under balanced selection. The common 4R allele appears to be the ancestral allele, with the 7R allele being a much younger allele. All rare variants appear to be recombination/mutation products of these common 4R and 7R alleles (FIG. 4 and above). For example, the 2R allele likely has both a 4R and 7R origin. Hence, simple 7R(+) and 7R(−) categories may not be appropriate divisions, and one should entertain other potential groupings. In particular, one might hypothesize that any amino acid alteration from the conserved ancestral 4R(1-2-3-4) haplotype might lead to altered biochemistry/phenotype. Tests of this hypothesis would group individuals as 4R/4R versus non-4R/4R for purposes of hypothesis testing.

What does the DRD4 /ADHD association mean? We have speculated that the very traits that may be selected for in individuals with a DRD4 7R allele may predispose behaviors that are deemed inappropriate in the typical classroom setting and hence diagnosed as ADHD. This environmental mismatch hypothesis (Jensen, P S, Mrazek, D, Knapp, P K, Steinberg, L, Pfeffer, C, Schowalter, J, and Shapiro, T. Evolution and revolution in child psychiatry: ADHD as a disorder of adaptation. J Am Acad Child Adolesc Psychiatry 1997; 36, 1672-1679) has testable predictions, including the potential benefit of altered educational approaches. In this hypothesis, the DRD4 7R subset of individuals diagnosed with ADHD are assumed to have a different, evolutionarily successful behavioral strategy rather than a disorder. Alternatively, we also speculated that DRD4 7R, while selected for in human populations, could have deleterious effects only when combined with other genetic variants. This complex genetic model for ADHD also has testable predictions. One of the many important questions stemming from this hypothesis is the number and nature of these interacting genes. Is DRD4 7R one of only a few (or a few hundred) predisposing alleles?

The DRD4 7R/ADHD association is one of the most reproduced in complex behavioral disorders. However, the approximately two-fold risk associated with the DRD4 7R allele and ADHD has been described as “small”. The implication is that DRD4 7R is but one of many predisposing alleles (a classic QTL; Lynch, M and Walsh, B. Genetic analysis of Quantitative traits (Sinauer Associates, Inc. Sunderland, M A) 1998), and indeed may be only a “modifier” of yet undiscovered predisposing genes. Certainly, this is a possibility. However, while a two-fold risk may be considered small in some contexts, this risk needs to be put in the perspective of observed DRD4 allele frequencies and the predictions of the CVCD hypothesis (FIG. 5).

In the populations of predominantly European ancestry used in most investigations of the DRD4/ADHD association, the allele frequency of DRD4 7R is approximately 12-15%. Therefore, even if the presence of a DRD4 7R allele was a necessary predisposing condition for ADHD (i.e., 100% of ADHD probands had at least one copy of this allele), and assuming Hardy-Weinberg equilibrium, the increase in observed frequency (and relative risk) would be only 3.6 fold (FIG. 5). If only half of ADHD is “caused” by DRD4 7R, then the increase in observed frequency would be 1.8 fold. Common alleles associated with a particular disorder, then, can only exhibit modest increases in allele frequency in affected individuals, and hence have modest relative risks (i.e., small λ). Most current genome scans of complex genetic disorders, including one for ADHD, would not have detected genomic regions with λ<2-3.

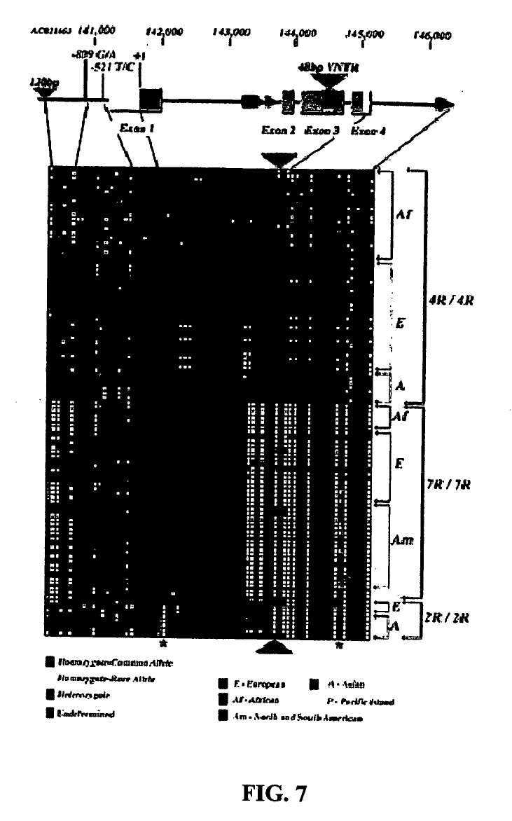

Are λ values less than 2-3 of little significance? Do they imply that the associated allele has little impact on the disorder? On the contrary, they are exactly of the magnitude one expects if the CVCD hypothesis is correct. Likewise, the RVCD model also predicts modest relative risks, if one sums the contributions of all variants in a single gene (FIG. 5). It is informative to propose a simple model for ADHD based on the CVCD hypothesis and the DRD4 7R association (FIG. 7). Unlike rare disorders like Huntington Disease, where the disease allele is rare and the allelic relative risk is large (>5,000 fold, FIG. 7), what if alleles predisposing to ADHD are common in the population? FIG. 7 outlines one such model, in which three different dominant alleles (designated DRD4 7R, b, c in three different genes) interact to predispose to the disorder. In this model, each of these alleles is at polymorphic frequency (0.05-0.12), and it is assumed that any two of them in combination predispose to ADHD. In such hypothetical interacting gene systems, any of the three “disease” alleles (DRD4 7R, b, or c) could also be described as “modifier” alleles, since their presence or absence effect the “penetrance” of the other alleles. Such interacting genetic systems should be common, since most gene products are part of multiprotein assemblies or biochemical pathways. Obviously, many other models could be proposed, involving recessive alleles, additional genes, etc. For example each predisposing “allele” could be many rare alleles (the RVCD model, FIG. 5), that in total have a frequency of 0.05. However, the model proposed in FIG. 7 is one of the simplest in which interacting alleles are neither necessary nor sufficient. In this example, approximately 5% of individuals would have one of the hypothesized predisposing genotypes [(DRD4 7R/x)(b/x), (DRD4 7R/x)(c/x), (b/x)(c/x)], approximately the observed incidence of ADHD (FIG. 7). None of the predisposing alleles would be either necessary or sufficient to “cause” ADHD. None of the hypothetical predisposing alleles would have a high λ (2-4 fold relative risk, FIG. 7), and none would likely be detected with genome scans of typical size. Yet according to this model, these are the predisposing alleles that are the object of our search. Similar conclusions could be reached for a variety of other likely models.

What can be concluded from such models? The observed two-fold increase in DRD4 7R allele frequency in ADHD probands is approximately 54% of the maximum possible (if all ADHD is genetic and related to DRD4 7R). As discussed above, this estimate modestly underestimates the relative risk, since rare 7R derivatives, as uncovered in this study, would not have been identified in prior work. The observed risk is approximately 87% of the maximum possible if 50% of ADHD has a nongenetic cause. If one assumes that ADHD predisposition is related to many different genes/alleles, such values for a single allele are, in fact, unusually high. We conclude, therefore, that the observed DRD4 7R-allele/ADHD association is not “small”, but is of a magnitude quite surprisingly high. It suggests that this allele is associated with a minimum of 25%-50% of the observed cases of ADHD. It further suggests that as few as one or two other common alleles in other genes, in combination with DRD4 7R (FIG. 7), could account for most of the disorder.

### The Genetic Architecture of Selection at the Human Dopamine Receptor D4 (DRD4) Gene Locus

Associations have been reported of the 7-repeat (7R) allele of the human dopamine receptor D4 (DRD4) gene with both the personality trait of novelty seeking and attention deficit/hyperactivity disorder. Above, based on the unusual DNA sequence organization of the DRD4 7R VNTR, we proposed that the 7R allele originated as a rare mutational event that increased to high frequency by positive selection (see also, Ding et. al., Proc. Natl. Acad. Sci. USA 99, 309-314, 2002). We have now resequenced the entire DRD4 locus from 103 individuals homozygous for 2R, 4R or 7R variants of the VNTR, a method developed to directly estimate haplotype diversity. DNA from individuals of African, European, Asian, North and South American and Pacific Island ancestry were used. 4R/4R homozygotes exhibit little linkage disequilibrium (LD) over the region examined, with more polymorphisms observed in African DNA samples. In contrast, the evidence for strong LD surrounding the 7R allele is dramatic, with all 7R/7R individuals (including those from Africa) exhibiting the same polymorphisms at most sites. By intra-allelic comparison at 18 high frequency polymorphic sites spanning the locus, we estimate that the 7R allele arose at the time of the “out of Africa” human exodus (approximately 42,500 years ago). Further, the pattern of recombination at these polymorphic sites is that expected for selection acting at the 7R VNTR itself, rather than at an adjacent site. We propose a model for selection at the DRD4 locus consistent with these observed LD patterns and the known biochemical and physiological differences between receptor variants.

The human dopamine receptor D4 (DRD4) gene, located near the telomere of chromosome 11p, exhibits an unusual amount of expressed polymorphism (Lichter, J. B., Barr, C. L., Kennedy, J. L., Van Tol, H. H. M., Kidd, K. K. and Livak, K. J. (1993) Human Molecular Genetics 2, 767-773; Ding, Y. C., Chi, H. C., Grady, D. L., Morishima, A., Kidd, J. R., Kidd, K .K., Flodman, P., Spence, M. A., Schuck, S., Swanson, J. M., et al. (2002) Proc. Natl. Acad. Sci. USA 99, 309-314; Grady, D. L., Chi, H. C., Ding, Y. C., Smith, M., Wang, E., Schuck, S., Flodman, P., Spence, M. A., Swanson, J. M., and Moyzis, R. K. Mol Psychiatry 8, 536-545). Much of this variation is the result of length and single nucleotide polymorphism (SNP) changes in a 48 bp tandem repeat (VNTR) in exon 3, encoding the third intracellular loop of this D2-like receptor. Alleles containing two (2R) to eleven (11R) repeats are found, with over 67 different haplotype variants uncovered to date. The three most common 2R, 4R, and 7R variants, however, represent over ninety percent of the observed allelic diversity. In most geographical locations, the 4R allele is the most common, while 2R and 7R allele frequency varies widely (Chang, F.-M., Kidd, J. R., Livak, K. J., Pakstis, A. J., and Kidd, K. K. (1996) Hum. Genet. 98, 91-101).

The functional significance of these length/sequence changes in the DRD4 protein, in a region that couples to G proteins and mediates intercellular cAMP levels, has been documented (Jovanovic, V., Guan, H. C., and Van Tol, H. H. M. (1999) Pharmacogenetics 9, 561-568; Oak, J. N., Oldenhof, J., and Van Tol, H. H. M. (2000) European J Pharmacology 404, 303-327). In particular, the 7R variant exhibits a blunted ability to reduce cAMP levels in comparison to the common 4R variant. The DRD4 protein is expressed in a number of brain regions, with high level expression in the prefrontal cortex, thought to be involved in cognition, attention and other higher brain functions. Significantly, DRD4 knockout mice display better performance on complex motor tasks, are supersensitive to cocaine, ethanol and methamphetamine, and exhibit reduced exploration of novel stimuli (Rubinstein, M., Phillips, T. J., Bunzow, J. R., Falzone, T. L., Dziewczapolski, G., Zhang, G., et al. (1997) Cell 90, 991-1001; Dulawa, S. C., Grandy, D. K., Low, M. J., Paulas, M. P., and Geyer, M. A. (1999) J Neurosci 19, 9550-9556). Taken together, these results are consistent with the proposal that DRD4 receptors act as inhibitors of neuronal firing, especially in the prefrontal cortex.

Based on these biochemical and physiological observations, a number of investigations have looked for associations between particular alleles of this highly variable gene and behavioral phenotypes (Swanson, J., Deutsch, C., Cantwell, D., Posner, M., Kennedy, J., Barr, C., Moyzis, R., Schuck, S., Flodman, P., and Spence, M. A. (2001) Clinical Neuroscience Research 1, 207-216; Faraone, S. V., Doyle, A. E., Mick, E., and Biederman, J. (2001) Am J Psychiatry 158, 1052-1057; Klugar, A. N., Siegfried, Z., and Ebstein, R. P. (2002) Mol Psychiatry 7, 712-717). While some studies have suggested that the DRD4 7R allele might be associated with the personality trait of novelty seeking, the most reproduced association is between the 7R allele and attention deficit/hyperactivity disorder (ADHD). ADHD is the most prevalent disorder of childhood (approximately 5% incidence), defined by symptoms of developmentally inappropriate inattention, impulsivity, and hyperactivity. The approximately two fold greater prevalence of the DRD4 7R allele in ADHD probands (1=1.9), calculated from a recent metaanalysis, indicates that this allele is associated with a significant fraction (25%-50%) of the attributable genetic risk for the disorder.

We have shown above by DNA resequencing/haplotyping of 600 DRD4 VNTRs, representing a worldwide population sample, that the origin of most haplotype variants could be explained by simple one-step recombination/mutation events. In contrast, the 7R allele is not simply related to the other common alleles, differing by greater than 6 recombinations/mutations. This unusual sequence architecture of the 7R VNTR, suggesting it arose as a rare mutational event, led to exploratory measures of linkage disequilibrium (LD) between the 4R and 7R alleles. Large discrepancies between allele ages estimated from low intra-allelic variability and high population frequency are taken as evidence that selection has increased the frequency of an allele beyond that expected by chance (Slatkin, M. and Rannala, B. (2000) Ann. Rev. Genomics Hum. Genet. 1, 225-249; Tishkoff, S. A., Varkonyl, R., Cahinhinan, N., Abbes, S., Argyropoulos, G., Destro-Bisol, G., et al. (2001) Science 293, 455-462; Sabeti, P.C., Reich, D. E., Higgins, J. M., Levine, H. Z. P., Richter, D. J., Schaffner, S. F., et al., (2002) Nature, 419, 832-837). Strong LD was found between the 7R-allele and four surrounding DRD4 polymorphisms, suggesting this allele is significantly “younger” than the common 4R-allele. Our preliminary estimates placed the origin of the DRD4 7R allele at approximately 40,000 years ago, a time of major human expansion out of Africa and the appearance of radical new technology (the upper Paleolithic) (Harpending, H. and Rogers, A. (2000) Annu. Rev. Genomics Hum. Genet. 1, 361-385; Ingman, M., Kaessmann, H., Paabo, S., and Gyllensten, U. (2000) Nature 408, 708-713; Underhill, P. A., Shen, P., Lin, A. A., Jin, L., Passarino, G., Yang, W. H., Kauffman, E., Bonne-Tamir, B., Bertranpetit, J., Francalacci, P., et al. (2000) Nature Genetics 26, 358-361). We speculate that these events and the appearance and selection for the DRD4 7R allele may be related.

If the DRD4 7R allele arose recently and underwent strong positive selection, why is it now disproportionately represented in individuals diagnosed with ADHD? One possibility is that an adjacent polymorphism in strong LD with the 7R VNTR is actually 1) associated with ADHD, or 2) the target of selection. We have argued that selection for an adjacent site was unlikely, given the distinct and unusual DNA sequence organization of the DRD4 7R allele itself, but due to the high density of SNPs in the human genome it remained a possibility. Obviously, even if the DRD4 VNTR is the site of selection, strong LD in the region could have carried an adjacent ADHD predisposing polymorphism along with it. Such “hitch-hiking” events should be common, again given the high density of SNPs in human DNA (The International SNP Map Working Group. (2001) Nature 409, 928-933).

Alternatively, however, the biochemical and physiological properties of the DRD4 protein discussed- above suggest -a more direct relationship. We- have proposed that the 7R/ADHD association is an example consistent with the Common Variant-Common Disorder hypothesis (Risch, N. and Merikangas, K. (1996) Science 273, 1516-1517), in which common predisposing alleles result in deleterious effects only when combined with other environmental/genetic factors. We have speculated that the very traits that may be selected for in individuals with a DRD4 7R allele may predispose behaviors that are deemed inappropriate in the typical classroom setting and hence diagnosed as ADHD. In this environmental mismatch hypothesis (Jensen, P.S., Mrazek, D., Knapp, P. K., Steinberg, L., Pfeffer, C., Schowalter, J., et al. (1997) J Am Acad Child Adolesc Psychiatry 36, 1672-1679), the DRD4 7R subset of individuals diagnosed with ADHD is assumed to have a different, evolutionarily successful behavioral strategy rather than a disorder. It is also possible, however, that DRD4 7R, while selected for in human populations, could have deleterious effects only when combined with other genetic variants.

In order to clarify some of these issues, we report an extensive analysis of polymorphisms surrounding the DRD4 VNTR by genomic resequencing. Remarkably, we show that the 7R allele exhibits strong worldwide LD, in geographic locations as diverse as sub-Sahara Africa and South American rainforests. By intra-allelic comparison at 18 high frequency polymorphic sites spanning the locus, we confirm that the 7R allele arose only 42,500 years ago. Further, the pattern of recombination at these sites is that expected for selection acting at the VNTR itself, rather than at an adjacent polymorphism.

Materials and Methods

Establishing Cell Lines and DNA Purification. Lymphoblastoid cell lines were established for all individuals. Methods for transformation, cell culture, and DNA purification have been described (above; see also, Ding, Y. C., Chi, H. C., Grady, D. L., Morishima, A., Kidd, J. R., Kidd, K. K., Flodman, P., Spence, M. A., Schuck, S., Swanson, J. M., et al., (2002) Proc. Natl. Acad. Sci. USA 99, 309-314; Grady, D. L., Chi, H. C., Ding, Y. C., Smith, M., Wang, E., Schuck, S., Flodman, P., Spence, M. A., Swanson, J. M., and Moyzis, R. K. Mol Psychiatry 8, 536-545; Chang, F.-M., Kidd, J. R., Livak, K. J., Pakstis, A. J., and Kidd, K. K. (1996) Hum. Genet. 98, 91-101). All individuals gave their informed consent before their inclusion in this study, which was carried out under protocols approved by the Human Subjects Committees at the participating institutions. The geographical/ethnic origins of the 103 individuals used in this study, grouped by genotype, are:

4R/4R, 20 African (11 Biaka, 3 Chaga, 3 Mboti, 2 Hausa, 1 African American), 24 European (11 unspecified European, 5 Irish, 3 English, 3 German, 1 Greek, 1 Italian), 7 Asian (5 Han Chinese, 2 Japanese);

7R/7R, 6 African (2 Biaka, 2 Hausa, 1 Chaga, 1 African American), 16 European (6 unspecified European, 3 European/Hispanic, 2 Irish, 1 Italian, 1 Druze, 1 Danish, 1 English, 1 German), 19 Americas (6 Karitiana, 5 Ticuna, 4 Maya, 4 Surui), 2 Pacific (Nasioi);

2R/2R, 3 European (2 unspecified European, 1 Russian), 6 Asian (5 Han Chinese, 1 Yakut).

Twenty (19%) of these individuals were ADHD Probands (3) (15 European, 4 Asian, 1 African), including one 2R/2R, fourteen 4R/4R and five 7R/7R genotypes. Primate DNA was obtained from 5 chimpanzee (Pan troglodytes), 5 bonobo (Pan paniscus), and 5 western lowland gorilla (Gorilla gorilla gorilla) individuals.

PCR amplification and DNA sequencing. The entire DRD4 allelic region was PCR amplified as three overlapping fragments (totaling 6.3 kb), which cover positions 140173 to 146480 in GenBank accession number AC 021663. The current Human Genome Project (HGP) assembly contains a 9 kb unordered fragment containing the DRD4 locus (from BAC RP11-49619) but the terminal DRD4 upstream region of this contig contains 1.9 kb of Alu DNA. Forward and reverse primers for these amplifications were 140173^{F1} (5′-GTGGTCGCAGACATCTTGG-3′) (SEQ. ID NO. 6), 142075^{R1} (5′-TAGACGAAGAGCGGCAGCA-3′) (SEQ. ID NO. 7, 142057^{F2} (5′-TGCTGCCGCTCTTCGTCTA-3′) (SEQ. ID NO. 8), 145072^{R2} (5′-ATGCTGCTGCTCTACTGGG-3′) (SEQ. ID NO. 9), 144901^{F3} (5′-CCTGCTGTGCTGGACGCCCT-3′) (SEQ. ID NO. 10), and 146480^{R3} (5′-TAGTCGGAGAAGGTGTCCTG-3′) (SEQ. ID. NO. 11). PCR amplification and excess primer and dNTP removal was as described above (see also, Ding, Y. C., Chi, H. C., Grady, D. L., Morishima, A., Kidd, J. R., Kidd, K. K., Flodman, P., Spence, M. A., Schuck, S., Swanson, J. M., et al. (2002) Proc. Natl. Acad. Sci. USA 99, 309-314; Grady, D. L., Chi, H. C., Ding, Y. C., Smith, M., Wang, E., Schuck, S., Flodman, P., Spence, M. A., Swanson, J. M., and Moyzis, R. K. Mol Psychiatry 8, 536-545). Additional primer sequences for forward and reverse sequencing of the DRD4 amplification products are available on our web site (www.genome.uci.edu). DNA cycle sequencing on ABI 3100 and 3700 automated sequencers was as described above (see also, Ding, Y. C., Chi, H. C., Grady, D. L., Morishima, A., Kidd, J. R., Kidd, K. K., Flodman, P., Spence, M. A., Schuck, S., Swanson, J. M., et al. (2002) Proc. Natl. Acad. Sci. USA 99, 309-314; Grady, D. L., Chi, H. C., Ding, Y. C., Smith, M., Wang, E., Schuck, S., Flodman, P., Spence, M. A., Swanson, J. M., and Moyzis, R. K. Mol Psychiatry 8, 536-545; Riethman, H. C., Xiang, Z., Paul, S., Morse, E., Hu, X.-L., Flint, J., Chi, H.-C., Grady, D. L., and Moyzis, R. K. (2001) Nature 409, 948-951).

Analysis of sequence data: Analysis of sequence data was aided by Phred, Phrap, Polyphred and Consed (Nickerson, D. A., To be, V. O., and Tayler, S. L. (1997) Nucleic Acids Res 14, 2745-2751).

Capture of individual genotypes/haplotypes into a database (SNPMAN). The collection and editing of SNPs into a relational database is done via an in-house software package we have designated SNPMAN (Grady, D. L., Chi, H. C., Ding, Y. C., Smith, M., Wang, E., Schuck, S., Flodman, P., Spence, M. A., Swanson, J. M., and Moyzis, R. K. Mol Psychiatry 8, 536-545). Visual displays of SNP data were performed using VG (visual genotyping) (Nickerson, D. A., Taylor, S. L., Weiss, K. M., Clark, A. G., Hutchinson, R. G., Steingard, J., et al. (1998) Nature Genet 19, 233-240). Information on all SNPs identified in this study is available on our web site (www.genome.uci.edu).

Protein modeling. DRD4 protein variants were modeled using the crystallographic structure of rhodopsin as a template (Filipek, S., Teller, D.C., Palczewski, K., and Stenkamp, R. (2003) Ann Rev Biophysics Biomol Structure 32, 375-397).

Allele age calculations. Allele age calculations were conducted by standard methods. Briefly:

t=[1/ln(1−c)] ln [(x(t)−y)/(1−y)], where t=allele age, c=recombination rate, x(t)=frequency in generation t, and y=frequency on normal chromosomes. We assumed the origin of the 7R-allele was on a specific 4R haplotype, and calculations utilized the extreme values of c determined from the telomeric recombination frequencies (including 11p) obtained by Kong et al (Kong, A., Gudbjartsson, D. F., Sainz, J., Jonsdottir, G. M., Gudjonsson, S. A., Richardsson, B., Sigurdardottir, S., Barnard, J., Hallbeck, B., Masson, G., et al. (2002) Nature Genet 31, 241-247) (2cM/Mb-4cM/Mb). For example, the T/C polymorphism at position 140,692 in the DRD4 consensus sequence is 3889 bp upstream of the VNTR, and hence c values ranging from 0.0000778 to 0.0001556 were used (from the average recombination rate per Mb times the VNTR-SNP distance). In all cases, the frequency on normal chromosomes (y) was assumed to be that observed on chromosomes obtained from African 4R/4R individuals. Similar results- were obtained using y obtained from the entire 4R/4R population sample. The frequency of the derived allelex(t) was that observed in the total population of 7R/7R individuals. For example, the T/C polymorphism at position 140,692 has y=5.3% (the percent of the C variant in African 4R/4R individuals) and x(t)=84.9% (the percent of the C variant in all 7R/7R individuals). For conversion from time t in generations to years, a generation time of 20 years was assumed.

Linkage Disequilibrium. Analysis and display of LD was conducted using the GOLD program (Abecasis, G. R., and Cookson, W. O. (2000) Bioinformatics 16, 182-183).

Results

The unusual nature of the sequence architecture of the DRD4 7R VNTR, suggesting it arose as a recent rare mutational event, led us to determine if differences in LD exist between the 4R and 7R alleles. We resequenced 6,307 bp of contiguous DNA surrounding the DRD4 VNTR from 103 individuals (1.5 Mb total), chosen from previous screenings as homozygous for the VNTR (4R/4R, 7R/7R, 2R/2R; FIG. 7). DRD4 loci from 51 4R/4R individuals, 43 7R/7R individuals and 9 2R/2R individuals were resequenced. 7R/7R and 2R/2R individuals were highly oversampled in comparison to their frequency in the population. This approach was developed as a direct and efficient method to estimate the haplotype diversity surrounding the putative ancestral 4R allele in comparison to the recent 7R allele. The resulting sequence data was processed by SNPMAN and polymorphisms displayed using VG.

FIG. 7 displays the polymorphism distribution of individuals grouped by genotype (4R/4R, 7R/7R, and 2R/2R) and geographic origin (African, European, etc.). Individuals were intentionally chosen from diverse populations. For example, the African samples represent 13 Biaka, 4 Chaga, 4 Hausa, 3 Mboti and 2 African American individuals (see Methods). Due to the wide variation in 7R allele distribution, our sample includes an abundance of 7R/7R individuals of North and South American ancestry and none from Asia, where the 7R allele frequency is only 0.01 (FIG. 7). Our 4R/4R sample intentionally included a large fraction (39%) of individuals of African ancestry (FIG. 7), in order to estimate the “ancestral” frequency of polymorphisms (see below).

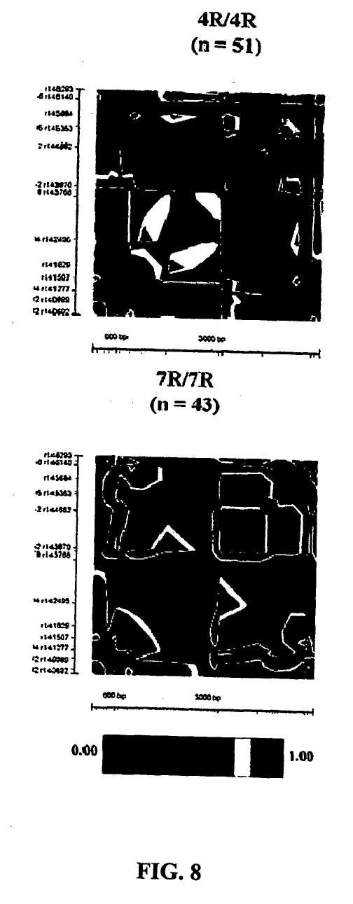

Not including VNTR variants, a total of 70 SNPs/polymorphisms were detected (on average, one per 90 bp), many at low frequency (FIG. 7). As expected, most of these low abundance SNPs were not in current databases. As can be seen in FIG. 7, the polymorphism spectral distribution of the 4R/4R homozygotes exhibits little LD over the region examined. In addition, twenty-eight percent (20/70) of the observed SNPs were found only in African samples (FIG. 7). These results are consistent with many studies on other genomic regions, and likely reflect the “fingerprint” of an out of Africa expansion of modern humans and a genetic bottleneck in European and Asian populations. FIG. 8 shows a graphical display of LD for the same 4R/4R data, using the GOLD program. GOLD displays all pairwise LD values as a color gradient aligned with the linear DNA sequence. As can be seen in FIG. 8, there is little LD above the 0.6 value expected at these close (<6 kb) distances.

In contrast, the evidence for strong LD surrounding the 7R allele is dramatic, with most 7R/7R individuals exhibiting the same polymorphisms at most sites (FIGS. 7 and 8). All 7R alleles, including those from African populations, exhibit the same strong LD. By resequencing this same genomic region in 15 primate genomes (Chimpanzee, Bonobo, and western lowland Gorilla), the likely ancestral SNP could be determined unambiguously for most SNP pairs. Seventy-six percent (13/17) of the most common variants (Table 5) were inferred to be ancestral in origin, with one SNP (144,842) having both variants in primate DNAs. Four of the most common variants in the population were “human specific” (Table 5). Forty-one percent (7/17) of the observed polymorphisms in tight LD with the 7R VNTR were the rarer human specific SNPs (FIG. 7 and Table 5).

Table 5. Calculated allele age for DRD4 7R. Eighteen polymorphisms in the DRD4 sequence are arranged in upstream to downstream order, and distance to the exon 3 VNTR is indicated. The most frequent polymorphism is listed first, in all cases the “ancestral” variant determined from primate DRD4 resequencing, except for the four noted with an asterisk in which the less common variant is ancestral. The frequency of the common polymorphism in African 4R/4R individuals and all 7R/7R individuals is given. All values were obtained from the data displayed in FIG. 7, except for polymorphisms 140,438, 144,842 and 144,862 which were obtained from a much larger sample set (over 2,000 individuals). Asterisks indicate “human specific” SNPs tightly linked to the 7R allele. Allele age was calculated from the extreme values of telomeric recombination reported in Kong et al. (Age1=4cM/Mb and Age2=2cM Mb) using standard methods. The average value obtained from all polymorphisms is 42,500 years (average Age1 and Age2), with maximum likely limits of 20,000-65,000 years.

| Polmorphism | | Distance | 4R/4R | 7R/7R | Age1 | Age2 |
|---|---|---|---|---|---|---|
| 140,438 | L/S(120 bp)* | 4143(−) | 61.9% | 90.8%* | 33,361 | 66,715 |
| 140,582 | G/del | 3999(−) | 95.0% | 13.9%* | 19,175 | 39,557 |
| 140,692 | T/C | 3889(−) | 94.7% | 15.1%* | 22,269 | 44,653 |
| 140,892 | T/C | 3689(−) | 70.0% | 95.4% | 22,558 | 45,119 |
| 141,507 | C/T | 3074(−) | 73.7% | 97.7% | 14,884 | 29,771 |
| 142,426 | G/A | 2155(−) | 90.0% | 97.7% | 28,961 | 57,922 |
| 143,578 | A/del | 1003(−) | 78.4% | 98.80% | 28,493 | 56,989 |
| 143,766 | C/A* | 815(−) | 77.5% | 4.6% | 37,539 | 75,078 |
| 143,862 | G/del | 719(−) | 95.0% | 2.3%* | 17,037 | 34,075 |
| VNTR | | 0 | | | | |
| 144,842 | G/A | 261 | 88.7% | 1.6% | 34,865 | 69,731 |
| 144,862 | G/C | 281 | 87.9% | 1.6%* | 32,686 | 65,373 |
| 145,239 | del/G* | 658 | 43.8% | 2.3% | 31,637 | 63,221 |
| 145,295 | T/C | 714 | 79.0% | 2.3%* | 20,690 | 41,381 |
| 145,353 | del/G | 772 | 41.7% | 2.3%* | 27,615 | 55,181 |
| 145,684 | A/C* | 1103 | 69.4% | 3.5% | 23,457 | 46,915 |
| 146,041 | T/C | 1460 | 42.1% | 3.8% | 26,699 | 53,397 |
| 146,056 | T/C | 1475 | 57.9% | 96.3% | 26,989 | 53,966 |
| 146,293 | C/A | 1712 | 44.7% | 4.6% | 28,187 | 56,367 |
| | | | | Average | 26,505 | 53,078 |
| | | | | | SD = 6,112 | SD = 12,139 |

Only a single new high abundance SNP was found in the 7R alleles examined, located in the downstream region of the gene (146,033; asterisk in FIG. 7). The majority of individuals containing this SNP were of North or South American ancestry (Karitiana, Ticuna, Maya, and Surui), suggesting a possible New World origin. This SNP was not found in our African population samples, and was at low frequency in our European populations, which included some individuals with partial Hispanic (and likely North/South American) ancestry (FIG. 7).

One exception to the strong LD found at the DRD4 7R locus is in a small 288 bp region at the promoter (−809 to −521 in FIG. 7), where 8 tightly spaced SNPs are found at comparable frequencies in both 4R and 7R alleles. Five of these SNPs are clustered in a region of only 95 bp. It is difficult to understand this specific breakdown of LD at the promoter unless numerous gene conversion events/mutations/selections have occurred (Ardlie, K., Liu-Cordero, S. N., Eberle, M. A., Daly, M., Barrett, J., Winchester, E., Lander, E. S., and Kruglyak, L. (2001) Am J Hum Genet 69, 582-589). Similar high frequency variations are found in this region in the limited primate samples examined, including more extensive deletions in chimp and gorilla (data not shown). These highly variable SNPs (140,989-141,277) are not included in Table 5, their ancestral origin (above) cannot be determined, and they cannot be used in allele age determinations (below). Regardless of the mechanism of homogenization at this small DRD4 promoter region, the strong LD observed at the 7R allele continues upstream of this region, with 4 high frequency SNPs/polymorphisms in tight linkage with the VNTR (FIG. 7 and Table 5). Genotyping of other VNTRs associated with three DRD4 adjacent loci (PTDSS2, HRAS, and SCT) indicates that the region of strong LD surrounding the DRD4 7R allele extends for at least 50-100 kb (data not shown).

Interestingly, from the resequencing of a sample of 2R/2R homozygotes (FIG. 7), the 2R allele appears to be a recombination product between a 4R and a 7R allele, as we originally proposed. The 2R VNTR downstream region contains a polymorphism pattern identical to that found in 7R alleles, while the VNTR upstream region is more variable, suggesting more than a single origin for this proposed recombination (FIG. 7). Most of the examined 2R alleles, however, contain a unique SNP (142,115; asterisk in FIG. 7) in the first intron, found only in a single 4R individual of African ancestry, suggesting a common origin and expansion for these 2R alleles.

Calculations of allele age based on the relatively high worldwide population frequency of the DRD4 2R, 4R and 7R alleles suggest that these alleles are ancient (>300,000-500,000 years old). On the other hand, calculations of allele age based on the observed intra-allelic variability (Table 5) suggest the 7R allele is 10 fold “younger” (42,500 years). Such-large discrepancies between allele ages calculated by these two methods are usually taken as evidence that strong selection has increased the frequency of the allele to higher levels than expected by random genetic drift. The absolute values of these estimates are greatly affected by the assumptions used in their computations, for example the assumed recombination frequency. For the calculations in Table 5, we have used the extremes of estimates of recombination frequency observed for the telomeric regions of human chromosomes (including 11p). All 18 high frequency DRD4 SNPs used to estimate allele age yield comparable results (Table 5). This suggests that the average of these values (42,500 years) is a reasonable current estimate of the allele age for DRD4 7R, comparable to our prior estimate of 40,000 years based on only four adjacent SNPs (2). Using the extremes of assumed recombination frequency (Age1 and Age2), plus or minus the standard deviation, yields an estimate of the limits of allele age from 20,000-65,000 years (Table 5). The proposed origin of the 2R allele as a 7R allele derivative (FIG. 7) indicates that it must also be a young allele. The discrepancy between the observed high frequency of the 2R allele, especially in Asian populations, and it's likely recent origin (FIG. 7) suggests that it too has likely increased in frequency by positive selection.

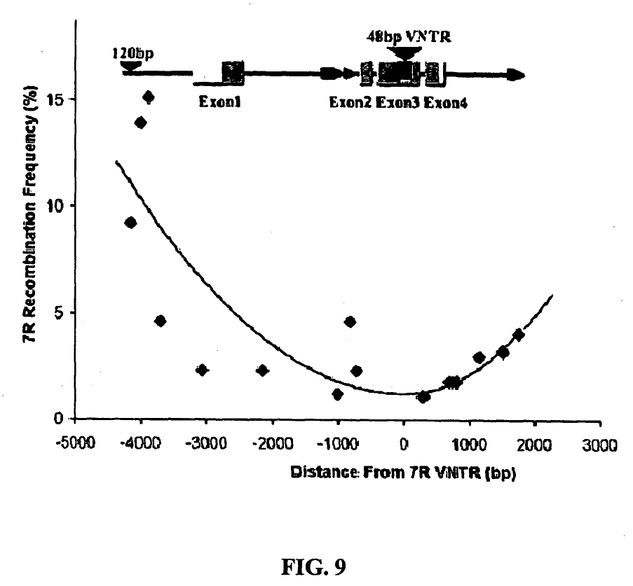

The data in FIG. 7 and Table 5 can also be used to test if the DRD4 VNTR itself, rather than an adjacent SNP, is the target of selection. Ideally, one should observe an increase in recombination (and lower LD) as distance from the selected polymorphism is increased. FIG. 9 plots distance from the DRD4 7R VNTR versus percent recombination. As expected if the DRD4 7R VNTR is the target of selection, the observed recombination is lowest near the VNTR, and increases with distance in both directions. Groupings based on splitting the population sampled based on any of the other 18 SNPs (for example splitting the sample based on the G/A 142,426 SNP rather than the 4R/7R VNTR) yielded largely random recombination patterns for adjacent SNPs (data not shown). While the observed recombination fraction is quite low, and there is significant scatter in the data (FIG. 9) these results support the hypothesis that the DRD4 7R VNTR is the target of selection.

Discussion

In this study, we have expanded our LD analysis of the DRD4 locus by resequencing the entire locus in 2R, 4R, and 7R homozygous individuals. This method was chosen as an accurate and efficient approach to determine the comparative LD of two alleles, requiring little statistical manipulation to infer haplotype differences. Using this approach (FIGS. 7 and 8), the pattern of LD surrounding the DRD4 4R allele is that expected for an ancient gene locus (300,000-500,000 years old), in which haplotype diversity is greatest in African populations, and more restricted outside Africa.

In contrast, the evidence for strong LD surrounding the 7R allele is dramatic (FIGS. 7 and 8). Such worldwide LD for a single selected human allele is remarkable. For example, in one of the best-characterized examples of selection in humans, the frequencies of low-activity alleles of glucose-6-phosphate dehydrogenase are highly correlated with the prevalence of malaria, yet many regional variants have been selected for. There is no worldwide “malaria resistant” variant, presumably because the introduction of agriculture 10,000 years ago (and the Plasmodium parasite) selected for independent regional mutations. By intra-allelic comparison at 18 high frequency polymorphic sites, we can estimate that the DRD4 7R allele arose approximately 42,500 years ago (with maximum likely limits of 20,000-65,000 years ago; Table 5). Further, the finding that forty-one percent of the 7R adjacent SNPs in tight LD are “human specific” (Table 5) argues for the derivation of this variant by mutation from the common human 4R allele, rather than importation from a related hominid lineage. Population bottlenecks and local admixture cannot explain the observed results. We propose, therefore, that the worldwide LD found for the DRD4 7R allele is a reflection of strong selection for this allele at the time of the major out of Africa exodus.

We suggested it is unlikely that selection for an adjacent gene can account for the proposed selection, given the distinct and unusual DNA sequence of the DRD4 7R VNTR itself. We have now shown that recombination frequency with adjacent SNPs is likely centered on the VNTR in 7R alleles (FIG. 9), suggesting that it is indeed the target of selection. Strong LD with the DRD4 7R allele can be detected at least 50-100 kb from the VNTR (near the PTDSS2, HRAS and SCT loci, data not shown). However, since the current HGP assembly in this subtelomeric region contains many gaps and ambiguous contig orders, it is impossible at present to refine these LD studies. Further work to define the limits of LD for this locus will help clarify both the estimates of allele age and the evidence for VNTR selection.

The breakdown of this strong LD at a small (288 bp) region at the promoter of the 7R allele is surprising (FIG. 7), and suggests that frequent gene conversion events/mutations/selections have occurred at this region. One can only speculate as to what mechanisms might be involved. It is especially intriguing, however, that this homogenization occurs at the promoter. Given that the CpG frequency at this site is not significantly higher than the remainder of this GC-rich gene, we suggest that high frequency gene conversion might explain this homogenization. Similar high frequency variations are found in primates, thus this region is a hotspot for such changes. Small hotspots for gene conversion have been proposed to exist at various loci in the human genome. The overall strong LD associated with the 7R allele continues upstream of this anomalous region (FIG. 7). Such data suggest using caution in inferring LD surrounding a particular genomic region based on a limited number of markers.

Extensive biochemical analyses of DRD4 protein variants have been conducted (5-7). The 7R protein has a blunted response for cAMP reduction, requiring a three-fold increase in dopamine concentration for reductions comparable to the 4R protein. This “suboptimal” response of the 7R allele to dopamine was hypothesized to underlie its association with the personality trait of novelty seeking and ADHD. It was suggested that the inhibitory neurons utilizing the DRD4 7R receptor would require increased dopamine for “normal” function (Swanson, J. M., Oosterlaan, J., Murias, M., Schuck, S., Flodman, P., Spence, M. A., Wasdell, M., Ding, Y. C., Chi, H. C., Smith, M., et al. (2000) Proc. Natl. Acad. Sci. USA 97, 4754-4759). Such increased dopamine levels were hypothesized to be provided by risk taking behavior (in the case of novelty seeking) or methylphenidate (in the case of ADHD). Methylphenidate is thought to act by binding to the dopamine transporter and raising the levels of dopamine at the synapse.

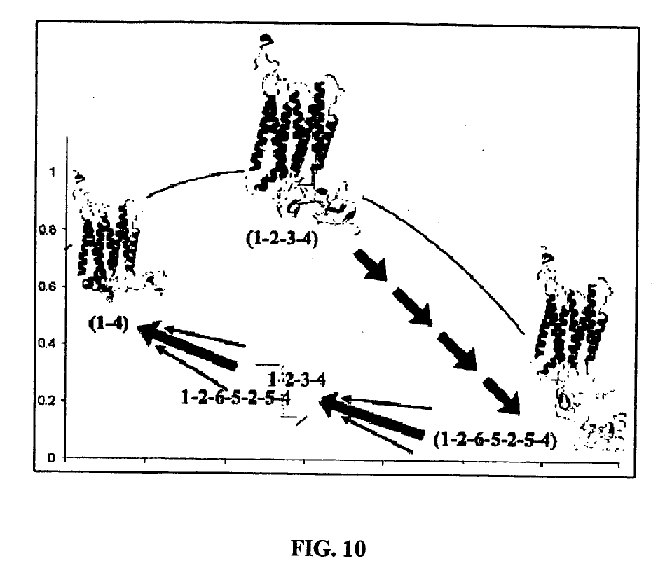

We propose a simple model integrating the known biochemical, physiological and genetic data regarding the common DRD4 alleles (FIG. 10). The 4R allele appears to be the dominant allele throughout most of human prehistory. This ancestral allele has the fewest amino acid changing variants, implying strong purifying selection. The 7R allele arose as a rare mutation approximately 42,500 years ago that significantly blunted the receptor's response to dopamine. This blunted response led to behaviors that were selected for in certain environments, and the two alleles (4R and 7R) coexisted in a balanced selection system, their relative frequencies varying by both chance and the environmental/cultural conditions. For example, it has been suggested that resourcedepleted, time-critical, or rapidly changing environments might select for individuals with “response ready” adaptations, while resource-rich, time-optimal, or little changing environments might select against such adaptations. We have speculated that such a “response ready” adaptation might have played a role in the out of Africa exodus, and that allele frequencies of genes associated with such behavior would certainly be influenced by the local cultural milieu (above; see also, Harpending, H. and Cochran, G. (2002) Proc. Natl. Acad. Sci. USA 99). Consistent with this “response ready” behavior hypothesis is the significantly better performance of DRD4 knockout mice on tests of complex coordination, and the observed faster reaction times exhibited by ADHD individuals with a 7R allele in comparison to non-7R individuals (Langley, K., Marshall, L., van den Bree, M., Thomas, H., Dphil, O., O'Donovan, M., and Thapar, A. (2003) Amer J Psychiatry, in press).

The genetic data suggest that most 2R alleles are 7R derivatives, and likely had limited (yet multiple) origins (FIG. 10). Interestingly, the 2R variant also has a blunted cAMP response, but one midway between the 4R allele and 7R allele (FIG. 10). Perhaps individuals with 2R alleles exhibit behaviors “intermediate” between those manifested by 4R and 7R alleles? This “non-linear” response (i.e., cAMP reduction capability is not linearly related to DRD4 VNTR repeat length) is consistent with the genetic evidence, and suggests a typical biochemical “optimum” strategy (FIG. 10). In this model, the 4R variant has been honed for hundreds of thousands of years to function optimally, while the new 7R and 2R variants are suboptimal yet confer a behavioral advantage in some environments. We propose that all three alleles are maintained in the population by balanced selection, their relative frequencies dependent on both chance and local selective pressures.

Such frequency dependent adaptive strategies are common, and are predicted by evolutionary game theory (Smith, J. M. Evolution and the Theory of Games. (1982) Cambridge: Cambridge University Press). A now classic example is the “rock-paper-scissors” color morphs in the side-blotched lizard (Sinervo, B. and Lively, C. M. (1996) Nature 380, 240-243). In this species, color is controlled by a single locus (OBY) that serves as a genetic marker for three different male behavioral strategies. Orange males usurp territory, blue males are mate guards, and yellow males are sneakers. Sneakers beat aggressive usurpers, mate-guards beat sneakers, and usurpers beat mateguards. Male competition drives cycles analogous to a rock-paper-scissors game, with all three strategies successfully reproducing (at varying frequencies) in the population.

While this speculative model (FIG. 10) is based on available genetic, biochemical, and physiological data, obviously only further work can test, refine and modify these ideas. The evidence for selection acting at the DRD4 locus is strong, however (FIGS. 7 and 9), and challenges us to determine the specific mechanism driving it. Regardless of the ultimate details, is it reasonable to think that a single gene variation can modify human behavior and be shaped by cultural diversity? We argue that just such single gene changes regulating complex social behavior have been identified in other organisms (Krieger, M. J. B. and Ross, K. G. (2002) Science 295, 328-332). We see no reason to think humans should be exempt from similar Darwinian selection (Darwin, C. The Descent of Man and Selection in Relation to Sex. (1871) London: J. Murry.), and suggest the exciting possibility that the DRD4 locus is a prime candidate for such gene-culture interactions.

Diagnostic Test for ADHD using DRD4 Probes.

The invention provides a method for testing patients for ADHD using probes derived from the DRD4 7R allele, or markers from within an area of strong linkage disequilibrium with the DRD4 7R allele. The invention provides a DNA oligomer comprising a DNA sequence complementary to DNA encoding the DRD4 7R allele, or markers from within an area of strong linkage disequilibrium with the DRD4 7R allele. Under appropriate hybridization conditions, such probes may be used to screen samples from individuals for the presence of the DRD4 7R allele, or a marker from within an area of strong linkage disequilibrium with the DRD4 7R allele. It should be readily apparent to those skilled in the art that a sequence complementary to the anti-sense strand of the DRD4 7R allele is also provided by the subject invention.

The DNA oligomer can be labeled with a detectable marker, such as a radiolabeled molecule, a fluorescent molecule, an enzyme, a ligand, or biotin. The labeled oligomer can then be utilized to detect the presence of the DRD4 7R allele, or a marker from withing an area of strong linkage disequilibrium with the DRD4 7R allele, so as to diagnose ADHD. This method comprises:

a) obtaining a tissue sample from the subject;

b) treating the sample so as to expose DNA present in the sample;

c) contacting the exposed DNA with the labeled DNA oligomer under conditions permitting hybridization of the DNA oligomer to any DNA complementary to the DNA oligomer present in the sample, the DNA complementary to the DNA oligomer containing the DRD4 7R allele or other marker within the region of strong linkage disequilibrium;

d) removing unhybridized, labeled DNA oligomer; and

e) detecting the presence of any hybrid of the labeled DNA oligomer and DNA complementary to the DNA oligomer present in the sample, thereby detecting the allele or other marker and diagnosing ADHD.

Alternatively, DNA isolated from samples taken from individuals can be amplified by PCR using primers directed to the DRD4 gene or other markers within the area of strong linkage disequilibrium, and sequenced to determine the presence of specific DRD4 alleles as described above.

All methods for detecting the presence of a specific DNA sequence in DNA isolated from an individual known to one of skill in the art are contemplated to fall within the scope of this invention. Thus, any method of detecting the presence of the DRD4 7R allele, or other marker in the region of linkage disequilibrium, is within the scope of this invention.

While this invention has been described in detail with reference to a certain preferred embodiments, it should be appreciated that the present invention is not limited to those precise embodiments. For example, the reagents and methods of the present invention include not just those specifically disclosed, such as specific identified alleles associated with ADHD, but also to any markers subsequently found by routine experimentation to fall with the area of strong linkage disequilibrium with the DRD4 alleles identified above. Rather, in view of the present disclosure which describes the current best mode for practicing the invention, many modifications and variations would present themselves to those of skill in the art without departing from the scope and spirit of this invention. The scope of the invention is, therefore, indicated by the following claims rather than by the foregoing description. All changes, modifications, and variations coming within the meaning and range of equivalency of the claims are to be considered within their scope.

What is claimed is:

1. Reagent useful for diagnosing attention deficit hyperactivity disorder (ADHD), comprising a polynucleotide corresponding to an allele of DRDR associated with individuals exhibiting ADHD.

2. The reagent of claim 1, wherein the polynucleotide corresponds to the DRD4 7R allele.

3. Reagent useful for diagnosing ADHD, comprising a polynucleotide corresponding to a marker the locus of which is within a block of linkage disequilibrium surrounding the DRD4 7R allele.

4. The reagent of claim 3, wherein the locus of the marker is within 100 kB of the DRD4 7R allele.

5. The reagent of claim 3, wherein the locus of the marker is within 50 kB of the DRD4 7R allele.

6. Reagent useful for diagnosing ADHD, comprising a pair of oligonucleotides corresponding to an allele of DRDR associated with individuals exhibiting ADHD.

7. The reagent of claim 6, wherein the pair of oligonucleotides corresponds to the DRD4 7R allele.

8. Reagent useful for diagnosing ADHD, comprising a pair of oligonucleotides corresponding to a marker the locus of which is within a block of linkage disequilibrium surrounding the DRD4 7R allele.

9. The reagent of claim 8, wherein the locus of the marker is within 100 kB of the DRD4 7R allele.

10. The reagent of claim 8, wherein the locus of the marker is within 50 kB of the DRD4 7R allele.

11. A method for diagnosing ADHD in an individual, comprising the steps of:
a) obtaining a tissue sample from the individual;
b) treating the sample so as to expose DNA present in the sample;
c) contacting the exposed DNA with a labeled DNA oligomer under conditions permitting hybridization of the DNA oligomer to any DNA complementary to the DNA oligomer present in the sample, the DNA complementary to the DNA oligomer containing the DRD4 7R allele;
d) removing unhybridized, labeled DNA oligomer; and
e) detecting the presence of any hybrid of the labeled DNA oligomer and DNA complementary to the DNA oligomer present in the sample, thereby detecting and diagnosing ADHD.

12. A method for diagnosing ADHD in an individual, comprising the steps of:
a) obtaining a tissue sample from the individual;
b) treating the sample so as to expose DNA present in the sample;
c) contacting the exposed DNA with a labeled DNA oligomer under conditions permitting hybridization of the DNA oligomer to any DNA complementary to the DNA oligomer present in the sample, the DNA complementary to the DNA oligomer containing a marker within a region of strong linkage disequilibrium to the DRD4 7R allele;
d) removing unhybridized, labeled DNA oligomer; and
e) detecting the presence of any hybrid of the labeled DNA oligomer and DNA complementary to the DNA oligomer present in the sample, thereby detecting and diagnosing ADHD.

13. A method for diagnosing ADHD in an individual, comprising the steps of:
a) obtaining a tissue sample from the individual;
b) providing an oligonucleotide complementary to the sense strand of the DRD4 gene;
c) providing an oligonucleotide complementary to the antisense strand of the DRD4 gene;
d) treating the sample so as to expose DNA present in the sample;
e) contacting the exposed DNA with the oligonucleotides under conditions permitting amplification of the DRD4 gene;
f) sequencing the product of the amplification; and
g) detecting the presence of the DRD4 7R allele in the sample, thereby detecting and diagnosing ADHD.

14. A method for diagnosing ADHD in an individual, comprising the steps of:
a) obtaining a tissue sample from the individual;
b) providing an oligonucleotide complementary to the sense strand of a marker sequence found in an area of strong linkage disequilibrium with the DRD4 7R allele;
c) providing an oligonucleotide complementary to the antisense strand of the marker sequence;
d) treating the sample so as to expose DNA present in the sample;
e) contacting the exposed DNA with the oligonucleotides under conditions permitting amplification of the marker sequence;
f) sequencing the product of the amplification; and
g) detecting the presence of the marker sequence in the sample, thereby detecting and diagnosing ADHD.


SEQUENCE LISTING (11 sequences)

SEQ ID NO: 1 - DNA, 47 bp, Homo sapiens
cccgcgccccgcctcccccaggacccctgcggccccgactgtgcgcc

SEQ ID NO: 2 - DNA, 17 bp, Homo sapiens
tgggccgccgcattcgt

SEQ ID NO: 3 - DNA, 21 bp, Homo sapiens
ggtgggtgtatcgccgaggga

SEQ ID NO: 4 - DNA, 21 bp, Homo sapiens
cgtactgtgcggcctcaacga

SEQ ID NO: 5 - DNA, 21 bp, Homo sapiens
gacacagcgcctgcgtgatgt

SEQ ID NO: 6 - DNA, 19 bp, Homo sapiens
gtggtcgcagacatcttgg

SEQ ID NO: 7 - DNA, 19 bp, Homo sapiens
tagacgaagagcggcagca

SEQ ID NO: 8 - DNA, 19 bp, Homo sapiens
tgctgccgctcttcgtcta

SEQ ID NO: 9 - DNA, 19 bp, Homo sapiens
atgctgctgctctactggg

SEQ ID NO: 10 - DNA, 20 bp, Homo sapiens
cctgctgtgctggacgccct

SEQ ID NO: 11 - DNA, 20 bp, Homo sapiens
tagtcggagaaggtgtcctg